(19) United States
(12) Patent Application Publication
(10) Pub. No.: US 2026/0354684 A1
(43) Pub. Date: Nov. 12, 2026

(54) SYSTEM AND METHOD FOR EYE EXAMINATION

(71) Applicant: SLITLED LTD, Haifa (IL)

(72) Inventors: Michael NARODIZKY, Rehovot (IL); Benjamin HALEVY, Kiryat Tiv'on (IL); Zvi YONA, Karkur (IL); Avihu Meir GAMLIEL, Pardes Hana (IL)

(21) Appl. No.: 19/491,902

(22) PCT Filed: Jun. 24, 2024
(86) PCT No.: PCT/IL2024/050613

§ 371 Date: Dec. 10, 2025

Related U.S. Application Data
(60) Provisional application No. 63/523,054, filed on Jun. 24, 2023.

(51) Int. Cl. A61B 3/15; A61B 3/00; A61B 3/113; A61B 3/14; G06T 7/11; G16H 40/67; H04N 13/156; H04N 13/254; H04N 23/56; H04N 23/60; H04N 23/611; H04N 23/66; H04N 23/67; H04N 23/698; H04N 23/74; H04N 23/81

(52) U.S. Cl. CPC A61B 3/152; A61B 3/0025; A61B 3/113; A61B 3/145; G06T 7/11; G16H 40/67; H04N 13/156; H04N 13/254; H04N 23/56; H04N 23/611; H04N 23/64; H04N 23/66; H04N 23/67; H04N 23/698; H04N 23/74; H04N 23/81; G06T 2207/10012; G06T 2207/20132; G06T 2207/30041

(57) ABSTRACT

Some embodiments relate to a system for eye examination including a semi-autonomous system performing examination sessions of the eye, and a control system. The semi-autonomous system includes an illumination device including optical projector(s) operable to automatically project a sequence of illumination spots of certain shape on a plurality of locations across an eye region during the examination session; and imaging device(s) operable to acquire images of the eye being illuminated and generate image data in association with the plurality of locations. The control system is responsive to input data comprising preset data indicative of an examination task to define operational data for the semi-autonomous system defining operational parameters of the examination session. The control system operates the imaging device(s) synchronously with respective optical projector(s) thereby enabling to minimize image deterioration factor in said image data and minimize time of the eye exposure to the illumination during the examination session.

### CROSS-REFERENCE TO RELATED APPLICATIONS

This application is a national phase filing under 35 C.F.R. § 371 of and claims priority to PCT Patent Application No. PCT/IL2024/050613, filed on Jun. 24, 2024, which claims the priority benefit under 35 U.S.C. § 119 of U.S. Provisional Patent Application No. 63/523,054, filed on Jun. 24, 2023, the contents of which are hereby incorporated in their entireties by reference.

### TECHNOLOGICAL FIELD

The present disclosure relates to eye examination techniques and is particularly useful in slit-lamp type eye examination.

### BACKGROUND AND BACKGROUND ART

About 90% of the cases of diseases causing severe visual impairment can be treated successfully to stop further deterioration or even improvement, in case of on time diagnosis and treatment. The growth of the world population, the increase in life expectancy resulting in rise of elderly population that suffers more from ophthalmic diseases, and the spread of various ophthalmic disease in the general population are all contributing to rapid growth in the demand for eye-care services.

The population is scattered across the world, including rural and peripheral areas. However, eye-physicians tend to concentrate in urban and metropolitan centers. In addition, limited availability of medical services affects numerous populations globally, primarily due to factors such as lack of eye-physicians, geo-political circumstances, financial constraints, and various other reasons.

All the above raised the demand for Tele-Ophthalmology and Tele-optometry solutions to address those issues. The demand was further amplified by epidemic outbreaks such as COVID19. As will be described below, there is no adequate solution in the art for a remotely controlled slit-lamp examination, mainly due to technological limitations.

Imaging of a tissue using projection systems is well known in the art. Examples of imaging and projecting light systems include a magnifier microscope used for detailed examination. These kinds of imaging and projecting systems produce an image or vision that gives detailed information on the examined object and its constructed specific layer.

One such system is the “Slit Lamp” device that is used for eye examination. This device serves as the primary examination tool of eye-physicians for almost a century. In this device, a slit of light is projected on the examined parts of the eye. The projected slit has sharp edges, and it curves on any structure it meets along its path. The slit of light is partially penetrating through translucent or transparent layers/tissues (such as tears film, conjunctiva, cornea, aqueous humor, lens, vitreous and the inner layers of the retina). It is partially reflected (and partially absorbed) from the non-transparent layers/tissues (adnexa, sclera, iris and the pigment epithelium layer of the retina).

The slit's reflections and refractions are observed by both eyes of the eye-physician via a binocular microscope. The sharp edges, curving on the different structures, assist the eye-physician to assess the three-dimensional structure of the examined parts of the eye. The binocular microscope further contributes to the three-dimensional interpretation of the eye physician by providing a stereoscopic view of the examined parts.

The slit of light is also useful in measurement of structures and findings sizes, depths, borders and proportions.

Furthermore, the slit of light helps the eye-physician to concentrate on the examined structure that is illuminated by the slit, against the darker background tissue.

As a result of all the above qualities of the slit, the brain of the eye-physician can interpret, analyze and differentiate between normal and pathological findings in the examined parts of the eye.

During the examination, the eye-physician is moving the slit across the eye, by physically moving certain optical components of the illumination source, while aiming and focusing the slit and the microscope on the various parts of the eye. This allows the eye physician to assess the overall status of the examined eye.

The eye physician can select various slits' attributes, such as: width, length, color, intensity and the illumination angle. This provides the eye-physician with additional clinical information, allowing a proper diagnosis of the findings.

According to the conventional technique, the slit lamp device must or should be operated by a highly skilled eye-physician (ophthalmologist or Doctor of Optometry) to aim and focus the slit of light and the binocular microscope, examining the region of interest and assessing the patient pathological and normal findings of the various organs of the eye. Thus, the eye-physician must or should engage with the patient through the entire examination and needs to document his own perspective of the situation without any visual reference data (there is no image to refer to). For posterior part examination, conducted via the pupil, the eye-physician must or should hold additional type of lenses between the microscope and the examined eye and to aim it in addition to aiming the microscope and the illumination, requiring even higher expertise from his side. The conventional slit lamp device is an adequate tool in a face-to-face examination setup.

### GENERAL DESCRIPTION

There is a need in the art for an improved system for slit-lamp eye examination of a patient that can be easily operated remotely and on-site even by a non-specialist, after only a short training, resolving the patient cooperation issue, due to short and non-blinding burst of illumination.

The above-described conventional approach suffers from the requirement for the eye-physician, the examined patient and the Slit Lamp device to be all located at the same location for every ophthalmic examination, as well as from lack of visual documentation and follow-up capabilities. Indeed, the conventional Slit Lamp examination technique was neither designed nor intended to be used remotely and thus cannot be used for a remote examination setup. The eye-physician typically needs to memorize the findings while exploring the eye, to document it textually, record it into a storage device, and submit a final report. Even more so, the conventional approach does not resolve the patient convenience problems by, e.g., shortening the “chair time”.

However, during the examination, a collaboration between the eye physician and the patient is needed due to the nature of the intense illumination, patient's sensitivity to it and uncomfortable feeling due to illumination into the eye lasting up to 2-3 minutes (in cases of pathology even more). The difficulty of achieving proper collaboration between the eye physician and the patient is one of the major drawbacks of current slit-lamp based examination technique.

“Digital Slit Lamp” techniques have been developed which are based on the use of a traditional Slit Lamp, equipped with digital camera/s, which allow documentation, visual follow-ups, and patient education capabilities. The videos/images acquisition is performed by an ophthalmic professional, and the acquired information can be used for getting additional opinion and for consultation between eye-physicians, thus allowing some degree of ophthalmic telemedicine (remote examination). However, in most conventional setups requiring remote examination or consultation, the local operator is a non-physician thus not qualified for acquiring high-quality examinations on camera (video/images).

In many cases the local operator is unqualified to aim and focus the illumination and the camera on various parts of the eye. Moreover, the local operator isn't medically trained to capture a specific structure or layer in the eye. Most importantly, the local operator is not qualified to medically interpret the observed, normal or pathological findings, thus is not capable of continuing the examination according to those findings, including selecting suitable parameters for the exam, like a trained eye-physician would do. Thus, it will be extremely difficult for the remote eye-physician to perform a proper diagnosis, based on the received videos/images.

Also known in the art are “Manually Remotely Controlled Slit-Lamps”, for example, including a “Digital Slit Lamp” equipped with motors on various motion axes and a motion controller, connected to the web to thus allow for receiving commands from the remote eye-physician while sending live stream video back to him. This way, “Manually Remotely Controlled Slit-Lamps” micromanagement, allow the remote eye-physician to conduct the examination synchronously from a different geographical location. The remote eye-physician can operate the examination procedure and its various parameters, such as illumination intensity or color in addition to motion control, while observing the examined eye on his screen.

However, there are significant limitations in this type of synchronous, real time, examination where the remote eye-physician is operating the motion of the system directly. Those limitations prevent a proper medical examination and diagnosis. Such limitations include:
(i) Eye movements—latencies present inherent limitations for a synchronous remote examination. Such latencies include bi-directional communication and high-quality image and video streaming and motor motion latencies.

Those latencies in combination with various eye movements, such as saccades, blinking, etc., prevent the remote eye-physician from aiming and focusing the slit of light and the microscope on the desired part of the eye. By the time the remote eye-physician's instruction to move or to focus on a certain desired location is accomplished, the eye has already moved and the desired part of the eye is no longer there. Therefore, the remote eye-physician is unable to aim on the desired part of the eye as it is done in the traditional slit lamp examination.
(ii) Distorted Images—in the traditional slit lamp examination, at every moment of the examination, the eye-physician sees sharply and in high-quality, only fragments of clear image, while the other parts are blurred, distorted, not in focus, or even unseen. Therefore, the eye-physician is aiming and focusing multiple times on various parts of the eye, gathering all the observed information to be reconstructed to a complete three-dimensional model of the examined eye in his mind.

All the described limitations are still present in the known techniques of “Manually Remotely Controlled Slit-Lamps”. Specifically, the duration of the exam using “Manually Remotely Controlled Slit Lamp” in many cases might be much longer than the duration of an in-person exam (that for itself takes 2-3 minutes on the average), the reasons being (i) due to latency and communication delays and (ii) since is it much harder for the physician to aim the system on the desired part of the eye. These difficulties result in much longer examination time (way above 2-3 minutes). In addition, due to the examination duration there is a degradation in the clinical quality of the exam, hence it might raise medicolegal issues. Specifically, since the duration of the exam is much longer due to the latency, both physician (trying to focus and track the eye) and patient (due to illumination intensity and exam duration) are getting tired, this results with degradation in the clinical quality of the exam. Thus, when observing a set of images on the screen, the eye physician is unable to compensate in his mind for all those distortions, as a bad image remains a bad image.

The present disclosure provides a novel technique for eye examination including a semi-autonomous system providing a shorter and more convenient eye examination for the local or remote eye-physician. As will be described further below, the eye examination technique of the present disclosure provides an advanced system for local or remote “slit-lamp based” ophthalmic examination with similar (or superior) to the known in the art medical quality, functionality, and mode of operation by the eye-physician.

Additionally, the present disclosure provides techniques for the capturing, storage, processing, image analyzing and displaying of the various tissue layers of the anterior and posterior segments of the eye and the adnexa. According to some of the aspects of the present disclosure, it is intended for use in ophthalmic medical devices by eye-professionals, including remote examination (telemedicine).

From the eye-physician's point of view, the examination technique utilizing the principles of the present disclosure allows concentration on the clinical aspects of the examination, rather than “micromanagement” of the examination device, especially in a remote setting.

Additionally, from the eye-physician's perspective, conducting the examination using the system for eye examination of the present disclosure, the data, viewing and examining and the experience are similar to the common practice, despite the different underlined technological concept. This allows the eye-physician to have a short learning-curve for using the eye examination system of the present disclosure.

Visual documentation of the medical findings enables a reliable and convenient follow-up. Furthermore, the unique approach of the present disclosure allows an asynchronous dynamic slit scan, controlled by the eye-physician, thus allowing to redo a previous examination.

For example, in order to perform a standard test, the system should be aimed at 22.5 degrees with slit projection of a width of about 300 micrometers, while the imaging part of the system is oriented fully frontal. Assume that in order to cover the eye, about 100 images should be taken. An asynchronous dynamic slit scan means that when triggering the capturing process, the system aims itself to the correct position in front of the eye (degree and focus) and all the ~100 images are taken automatically without any interference of neither the operator nor the physician. It is important to note that in this example the system stays stationary while capturing the ~100 images without any motor movements during the automatic set of acquisitions themself.

The resulting images obtained with the system of the present disclosure may be used for patient education and for collaborating information with the patient. It may also be used for public education such as the education of staff and professional students.

The resulting images obtained with the system of the present disclosure may be used for obtaining second opinion (clinical), share images, share clinical notes or marking on the images, collaborate suspected findings, or exam asynchronously. Moreover, the remote physician providing second opinion may take control over the system and set the operational parameters synchronously.

Solutions using still imaging (as opposed to video-based solutions), such as described in the present disclosure, can reduce the required communication bandwidth and allow for higher image quality and resolution. In addition, the technique of the present disclosure offers improved resilience to poor network connectivity, both thruput and latency.

The physician can view this series of images as a video stream and freeze on a specific image while preserving its full resolution without image compression. Moreover, the physician is able to tag findings, use digital zoom, scan images frame by frame back and forth.

In some embodiments, when saving the image acquisition parameters, it enables recapture the same scene, position and properties as in the previous exams, and enables comparing the same findings captured in two different exams, and visualizing them side by side (or overlap with transparency) while simultaneously scrolling\viewing the same slit in both images.

From the patient's perspective, undergoing the eye examination with the technique of the present disclosure is simpler and more comfortable at least because the technique of the present disclosure allows for significantly reducing the duration of an examination session during which the eye is exposed to high-intensity illumination while the patient needs to minimize the eye movement.

The combination of all the above-mentioned features of the technique for eye examination of the present disclosure, enables a reliable and efficient remote slit-lamp examination—for saving eyesight, improved eye-care services and result in time saving for both the physician, local operator and the patient.

Thus, according to one broad aspect of the present disclosure, it provides a system for eye examination including:
an illumination device including at least one optical projector, each of the at least one optical projector being configured and controllably operable to automatically project a sequence of illumination spots of a predetermined shape on a plurality of locations extending in a spaced-apart relationship across an eye region during the examination session of the eye; and
at least one imaging device, each of the at least one imaging device being configured and operable to acquire images of the eye including the plurality of locations being illuminated by the illumination spots during the examination session and generate image data in association with the plurality of locations, the image data being indicative of abnormalities with respect to each the locations within the eye region, and
   a control system including a manager utility and a controller, wherein the manager utility is configured and operable to be responsive to input data including preset data indicative of an examination task to define operational data for the semi-autonomous system being controlled by the controller, the operational data defining operational parameters of the examination session of the eye, the controller being configured to operate each of the at least one imaging device synchronously with a respective at least one optical projector thereby enabling to minimize image deterioration factor in the image data and minimize time of the eye exposure to the illumination during the examination session.

It should be noted that the input preset data may include (i) pre-stored data indicative of the examination task (associated with corresponding operational data for the semi-autonomous system) aimed at identifying a specific pathology and/or imaging a specific portion of the eye region, and/or (ii) dynamically provided input from a physician at a remote physician-related station.

The preset data may include list/sequences of patterns and/or various image acquisition parameters (illumination intensity, illumination type, spectra of illumination, focal conditions, etc.) to be used in eye exam, each in association with a specific pathology type and/or exam of a specific part (organ) of the eye region.

The preset data may be designed/configured for each clinical exam (to detect\analyze pathology) and may include pre-defined or dynamically updated data about one or more of the following: illumination angle, imaging angle, illumination intensity, slit shape, etc. In some embodiments, the preset includes parameters regarding the desired focal point location, specifying the exact spot in the eye where the system should focus.

The preset data may include data dedicated for detection of pathologies and/or data for the use of specific focal point mechanism.

It should also be noted that the term “semi-autonomous system” should be interpreted broadly, covering also a fully autonomous system. This is because the technique of the present disclosure in relation to the implementation of the eye exam can actually be implemented by a fully-autonomous equipment. For example, the system includes pre-stored information about the exam to be performed for a specific patient. The patient may thus enter his ID or the like and operate the system by pressing the operational button to start the exam. As will be described further below, the system will first perform the safety check to be sure the patient is in the proper registration position, etc.

In some embodiments, the eye examination system of the present disclosure is configured and operable to perform a general exam using a so-called “asynchronous mode”, being a fully automatic execution of a number (one or more) of predefined exams, in accordance with a respective exam-related preset, enabling automatic execution of a sequence of two or more exams across different regions of the eye and providing data indicative of broad overview of the eye's or eyes' condition. Such procedure can be initiated by personnel even without clinical training.

In some embodiments, the system is configured and operable to define the operational data based on selection of the exam configuration from the input data using synchronous or asynchronous mode. For example, the system performs selection from a list of exams (pre-stored data), intended to address specific part of the eye (eye region) or a suspected pathology. Thus, the set/number of predefined exams is provided being optimized to the selected eye part and/or pathology. This can be conducted with or without the eye physician's real-time observation. In another example, the selection is performed using synchronous mode, according to which the examination is conducted while the eye-physician is observing the eye in real-time (typically, at a remote station) enabling the physician preview of the expected image acquisition. This allows the physician to select and provide (as input data being part of preset) various examination parameters.

In some embodiments, the system is configured and operable to implement a so-called “hybrid mode” which combines the above-described synchronous and asynchronous modes.

It should be noted that the term “synchronous mode” of the system operation as one of the possible implementations should not be confused with the “synchronous” operation of the optical projector(s) and imaging device(s). The latter is kept in any of the system operational modes and presents synchronous image acquisition.

Thus, in some embodiments, the input data comprises preset data including a list of records in association with at least one of a specific pathology type, functionality and examination of a specific part of the eye region, where the records comprise a plurality of data records relating to various types of the operational data to be used in the examination session. Additionally, the input data may comprise data being received from a remote station of a physician being indicative of physician preview data of expected image acquisition during the examination session, based on physician's review of initial image data provided by the control system, where the physician preview data may comprise data indicative of one or more examination parameters. Alternatively or additionally, the input data comprises the preset data including data indicative of two or more examination tasks across different regions of the eye. The control system is adapted to operate the semi-autonomous system in a fully automatic mode enabling automatic execution of a sequence of two or more examination tasks, thereby providing data indicative of broad overview of the eye condition.

The semi-autonomous system of the present disclosure is preferably configured to avoid any movement of any physical element of the system after generation of an activation signal (e.g., in response to pressing a button) to start the exam session. In other words, the position of the system elements is stationary during the exam session.

The technique of the present disclosure utilizes eye exam by electronic scan of the eye region with images being projected. This allows projection of different or varying patterns on the eye region during the exam session (while keeping the “physical stationarity” of the system).

The system may include a number N (N≥1) of the optical projectors, as well as may include a number M (M≥1) of the imaging devices, where numbers N and M may or may not be the same.

For example a pair of optical projectors associated with (synchronously operated with) one or two imaging devices can be used for concurrently performing the exam of both eyes or for stereo imaging of one eye. Alternatively, or additionally, different optical projectors can be used configured to operate with different operational parameters (e.g., focal conditions, intensity, spectra, etc.) to concurrently or sequentially perform exams on different parts of the eye, e.g., anterior (cornea and/or lens and/or iris) and posterior (retina) parts.

In some embodiments, the at least one optical projector and the at least one imaging device are configured with an extended depth of field, such that the illumination spots being automatically projected on the plurality of locations are focused on the respective plurality of locations, and the image data is indicative of respective focused images of the locations.

In some embodiments, at least one optical projector and at least one imaging device are configured with an extended depth of field independently controlled by the controller.

More specifically, the extended depth of field may be selected to include multiple anterior segment parts of the eye and adnexa being in focus at once.

In some embodiments, the semi-autonomous system comprises a focusing mechanism configured and operable to initially focus at least one of the optical projector and the imaging device on a relatively high contrast part (such as iris) of the eye and then move the focus forward or backward toward a part (e.g., cornea) which is to be examined, based on a known distance between the high contrast part and the examined part.

For example, to set the focal point on the cornea, which is transparent in a healthy person, the system uses auto-focus on the iris (a high-contrast area) and then moves backward several millimeters to position the focal point near the cornea.

In some embodiments, the system analyzes the 3D structure of the eye parts relative to the examined area (for instance, by using the slits as structured light elements) to set the focal point on the desired area.

In some embodiments, the semi-autonomous system comprises an autofocus mechanism being automatically operated by the controller according to the operational parameters of the examination session of the eye. At least one of the optical projector and the imaging device may comprise the autofocus mechanism.

In some embodiment, the illumination system is configured to enable electronic iris and/or electronic zoom function.

Preferably, the predetermined shape of the illumination spot is a slit-like shape.

The at least one optical projector may be configured and controllably operable to automatically project the sequence of slit-like shaped illumination spots in a single-slit consecutive fashion, the image data including corresponding consecutive image data pieces, each image data piece including a single-slit image.

The at least one optical projector may be configured and controllably operable to automatically and simultaneously project at least a first array of spaced-apart slit-like shaped illumination spots in at least first multi-slit fashion; the image data including at least one first image, each being image of the at least first array of spaced-apart locations.

The optical projector may be operable to sequentially project the first array of the slit-like shaped illumination spots in the first multi-slit fashion and a second array of the slit-like shaped illumination spots in a second multi-slit fashion, on respective first and second arrays of the locations, wherein the first and second arrays of the locations are arranged in an interlaced fashion; the image data including the first and second images of respectively the first and second arrays of locations. The first and second locations may be partially overlapping.

In some embodiments, the at least one optical projector is configured and operable to automatically vary a spherical angle of projection of the illumination spot with respect to the eye, the image data including image data pieces of the respective locations in association with the data indicative of the spherical angle of projection.

In some embodiments, the system further comprises an imaging unit configured and operable to obtain a wide-field image of the eye, to thereby enable to align foreground image formed by the image data on a background image formed by the wide-field image of the eye.

In some embodiments, the system further comprises a registration assembly for registering a position of user's face during the examination session. For example, the control system further comprises an activation utility configured and operable to activate the semi-autonomous system to perform the examination session in response to a control signal indicative of a safety condition of the user's face being at the registered position; and/or the system comprises a sensing system configured and operable to monitor user's face position and generate corresponding sensing data to be analyzed in order to determine the user's face position with respect to the registered position for selective generation of the control signal.

The sensing system may be configured and operable to communicate the sensing data to a safety controller at a remote control station, where the sensing data is analyzed, and the control signal is selectively generated and communicated to the controller. For example, the system comprises a safety controller being responsive to the sensing data and configured and operable to analyze the sensing data and generate the control signal to the controller upon identifying the registered position of the user's face.

In some embodiments, the illumination device comprises a flash illuminator.

In some embodiments, the semi-autonomous system comprises an eye-aiming device automatically operated by the controller according to the operational parameters of the examination session of the eye.

In some embodiments, the semi-autonomous system comprises an eye tracking mechanism operated automatically by the controller during the examination session.

The semi-autonomous system may comprise one or more movement mechanisms for implementing controllable movement of one or more elements of the system; and may comprise a safety controller configured and operable to control the one or more movement mechanisms.

The at least one imaging device may be configured and operable to capture 3D images.

The at least one imaging device may comprise color or black and white sensors.

The at least one imaging device may comprise monocular and/or multi-spectral sensor.

The control system may further comprise an image processor utility configured and operable to analyze the image data and generate data indicative of the number of abnormalities in the respective number of locations.

The control system is preferably configured and operable to communicate data indicative of the image data to a remote control station for further processing to identify and analyze abnormalities in the eye.

The control system may comprise an image processor utility configured and operable to extract the single slit image out of each of the consecutive image data pieces of the respective locations, crop the extracted single slit image and paste the cropped extracted single slit image on top of a background image being a wide-field image of the eye. For example, the control system may comprise an image processor utility configured and operable to segment at least first multi-slit image into a set of corresponding single-slit images.

The image processor utility may be further configured and operable to paste each of the single-slit images, extracted from at least first multi-slit image, on top of a background image being a wide-field image of the eye.

The image processor utility may be configured and operable to generate data indicative of dynamic movement of the single-slit image over the eye, thereby enabling human pausing of the data being presented for a specific slit view.

The image processor may be configured and operable to utilize the image data to generate 3D reconstruction of the acquired data in the acquired images.

In the embodiments where the illumination device comprises at least one pair of the optical projectors, each pair of the optical projectors being associated with one or two imaging devices for performing stereo imaging of the eye and provide stereo image data of the eye region, the image processor may be configured and operable to utilize the stereo image data to generate a 3D reconstructed image out of two multi-slit images; identify at least one 3D single-slit image in the 3D reconstructed image; project at least one 3D single-slit image onto a 2D image thereby obtaining a 2D projected image including at least one single-slit image; extract at least one single slit image out of the 2D projected image; crop the extracted single slit image; and paste the cropped extracted single slit image on top of a background image being a wide-field image of the eye.

In some embodiments, the image processing utility is configured and operable to analyze the image data by applying algorithm techniques including one or more of artificial intelligence, image enhancement, image recognition, image sharpening and image restoration, encoding.

The image processing utility may be integral with at least one of the optical projector and the imaging devices.

In some embodiments, the control system (e.g., the manager utility) is configured for data communication with an image processor at a remote control station. The data communication may comprise data transfer using encrypted secure communication. The control system (e.g., the manager utility) may be configured and operable to generate compressed representation of the image data and transfer the compressed representation to the image processor at the remote control station.

The control system may be configured and operable to control automatic variation of one or more of the following operational parameters independently: dimensions of the illumination spot, illumination wavelength, illumination intensity, illumination angle, imaging angle, focus offset of the illumination and/or imaging device, eye steering, background illumination.

According to another broad aspect of the present disclosure, it provides a control system for managing and controlling eye examination by a semi-autonomous examination system including at least one optical projector operable to automatically project a sequence of illumination spots of a predetermined shape on a plurality of locations extending in a spaced-apart relationship across an eye region during the examination session of the eye, and at least one imaging device operable to acquire images of the plurality of locations during the examination session and generate image data in association with the plurality of locations; the control system being configured as a computerized system having data input and output utilities, memory, and an image processor utility, wherein the control system comprises:
a manager utility configured and operable to be responsive to input data including preset data indicative of an examination task to define operational data for the semi-autonomous system, the operational data defining operational parameters of the examination session of the eye, and
a controller being configured to utilize the operational data and operate at least one imaging device synchronously with the optical projector enabling to minimize image deterioration factor in the image data and minimize time of the eye exposure to the illumination during the examination session.

The control system may be configured as an electronic unit to be installed in the semi-autonomous examination system.

The control system may be configured and operable to communicate with a remote control station, the manager utility being responsive to instructions from the remote control station to update at least one of the preset data and the operational data.

The present disclosure, in its yet further aspect, provides a server control system, being a computerized system (including input and output utilities, memory, processor, etc.), connectable, via communication network, to subscriber eye examination systems of the type performing semi-autonomous eye examination procedures. Such server control system is configured and operable to perform model-based processing (AI-based processing) of input image data received from each subscriber system (being identified by the subscriber ID) and generate output data including (i) data indicative of suspected parts and observations in the eye region being examined to enable detection of abnormalities and/or pathologies; (ii) data indicative of abnormalities and/or pathologies in one or more parts of the eye region being examined; and/or (iii) quality level of the image data provided by the subscriber system; and/or (iv) data indicative of recommended preset data (e.g., operational parameter of the system, e.g., pattern(s) to be used) for examination session(s) to be performed by the specific subscriber system (with respect to specific part(s) of the eye and/or with respect to specific abnormality to be detected, and/or with respect to specific patient).

### BRIEF DESCRIPTION OF THE DRAWINGS

In order to better understand the subject matter that is disclosed herein and to exemplify how it may be carried out in practice, embodiments will now be described, by way of non-limiting examples only, with reference to the accompanying drawings, in which:

FIG. 1 illustrates pictorially the general principles of the eye examination technique of the present disclosure;

FIG. 2 shows by way of a block diagram the eye examination system of the present disclosure;

FIG. 3 shows an example of the eye examination system according to the principles of the present disclosure;

FIGS. 4A to 4C show examples of single-slit images obtained using the eye examination system of the present disclosure;

FIG. 5A shows a general art example of a single-slit image with marked pathological findings;

FIGS. 5B and 5C exemplify single-slit images obtained with the technique of the present disclosure enabling to extract pathology related data;

FIG. 6 shows a multi-slit image of multi-slit pattern projection on a real human eye;

FIG. 7 shows separation of a set of multi-slit images into single slits, their pasting on top of the background image, and example of the resulting single slit image;

FIGS. 8A and 8B exemplify the technique of the present disclosure utilizing projection of first and second sets of the illumination spots of a multi-slit shape onto, respective, first and second sets of locations on the eye, wherein the first and second locations are arranged in an interlaced fashion, and exemplify subsequent reconstruction of the multi-slit images into a set of single slit images;

FIG. 9 shows a stereo single-slit view of the examined eye, as displayed to the remote eye-physician; each image was generated by pasting the same slit on the background image, taken from its respective angle; the two images are interpreted by the eye-physician as a stereo single-slit image, similar to examination with the traditional, binocular slit lamp device; and

FIGS. 10A and 10B show by way of flow diagrams an asynchronous (store & forward) and synchronous (real time) examination flows, respectively, enabled by the system for eye examination of the present disclosure.

### DETAILED DESCRIPTION OF EMBODIMENTS

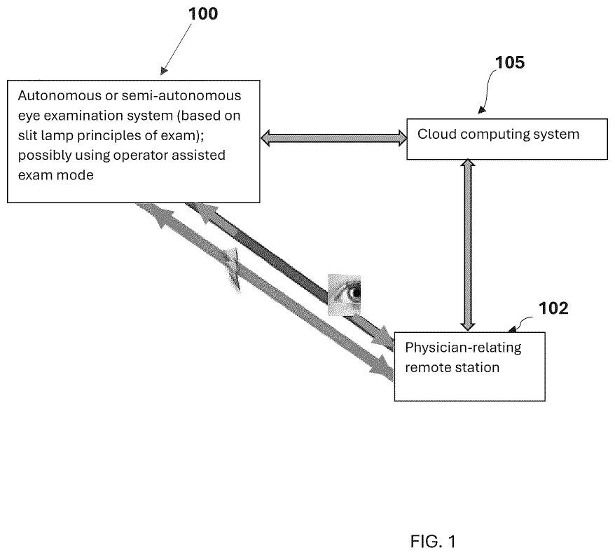

Reference is made to FIG. 1 pictorially illustrating the concept of the eye examination technique of the present disclosure providing a high confidence remote exam, with the exam quality being independent of operator's (non-physician) skills. The eye examination technique of the present disclosure enhances the detailed visual information displayed to the eye physician.

The technique of the present disclosure utilizes a real-time, remote-from-physician, automatic or semi-automatic eye examination system 100, configured and operable according to the present disclosure, that enables the eye physician, at a remote station 102, to handle several systems sites and enables a medical follow-up of specific patients either short or long time.

The examination system 100, as well as the physician-related station 102, can be also in data communication (via communication network and protocols of any known suitable type) with a cloud computing (server) system 105. As will be described below, the physician-related station 102 is used for viewing and remote parameters settings, while the heavy computation/data processing can be implemented by the cloud computing server 105. The server 105 can be used for exam management, exam's metadata and visual documentation, high level processing. Such server 105 can be an intermediate layer between system 100 components and physician-related system 102. The cloud computing can be implemented at either actual cloud server, remote PC, or even in local PC of the system 100 or system 102.

The automatic image acquisition performed by the system 100 may be initiated by a push of a button either locally by a local controller of system 100 or from the remote control station, e.g., at the physician station 102, thus eliminating a need for micromanagement (i.e., no direct and continuous micromanagement control of specific motors and system components is required. The slit moving and capturing micromanagement is done automatically by the system 100).

The system of the present disclosure advantageously reduces the discomfort of the examination, from the patient perspective, due to the short duration (e.g., <1 sec) of the exposure of patient's eye to the intense slit-lamp illumination, resulting in improved patient cooperation, further reducing the eye examination duration. The short exposure duration also resolves a major limitation of known in the art slit-lamp devices caused by eye movements of the patient (voluntary and non-voluntary) resulting in failure to aim to the desired part of the eye during the eye examination session. Attempts to implement a “Manually Remotely Controlled Slit Lamp” by connecting motors for controlling the various slit lamp movements and digital cameras for capturing video or images of the examined eye had limited success due to communication and motor movement latencies.

The short exposure duration, provided by the system of the present disclosure, is enabled by the novel optical setup of the semi-autonomous system of the eye examination system of the present disclosure. As will be described further below, such optical setup includes at least one illumination device configured and operable to automatically project a sequence of illumination spots of a predetermined shape, e.g., a slit-like shape, on the eye, while moving the location of the slit on the eye electronically. This movement of slit location (scan) is performed electronically, i.e., without a need for moving any light source components with motors, which unavoidably introduces motor movement latencies/artifacts. The system of the present disclosure also includes at least one imaging device, e.g., one or more cameras, which can be stationary during the examination session, i.e. during acquisition of the sequence of slit images. It is noted that in some embodiments, motors movements may be used, as long as the micromanagement is done automatically.

The projection of the illumination spots and the acquisition of the sequence of the respective images are performed synchronously and can be fast enough to provide continuous view of the examination area avoiding discontinuous slit coverage as a result of blinking, occlusion, pupil construction, etc., and to eliminate image blurring due to motion artifacts, e.g., avoiding eye movements as much as possible. It should be noted that the technique of the present disclosure allows for performing required image acquisition of the examination session, before the eye moves (including eye blinking) to another position thus causing discontinuity.

Thus, all required data for examination of the desired part of the eye can be obtained during a brief period of time allowing implementation of image freeze technology.

Specifically, the technique of the present disclosure allows a real time preview of the eye scene to the operator/remote physician, in order to aim the system to the required area, while by the moment the acquisition button is pressed, the previewed state is frozen and the images series are captured as stills (i.e., freeze image technology).

As will be described further below, the system of the present disclosure allows the physician to modify the capturing parameters, such as illumination angle, capturing angle, slit size (width, height, shape, orientation), slit intensity, etc., however, whenever the physician presses the capturing button, all the acquisition micromanagement is performed automatically by the system. The system enables independent control of viewing parameters such as zoom (optical and/or digital) and observation angle.

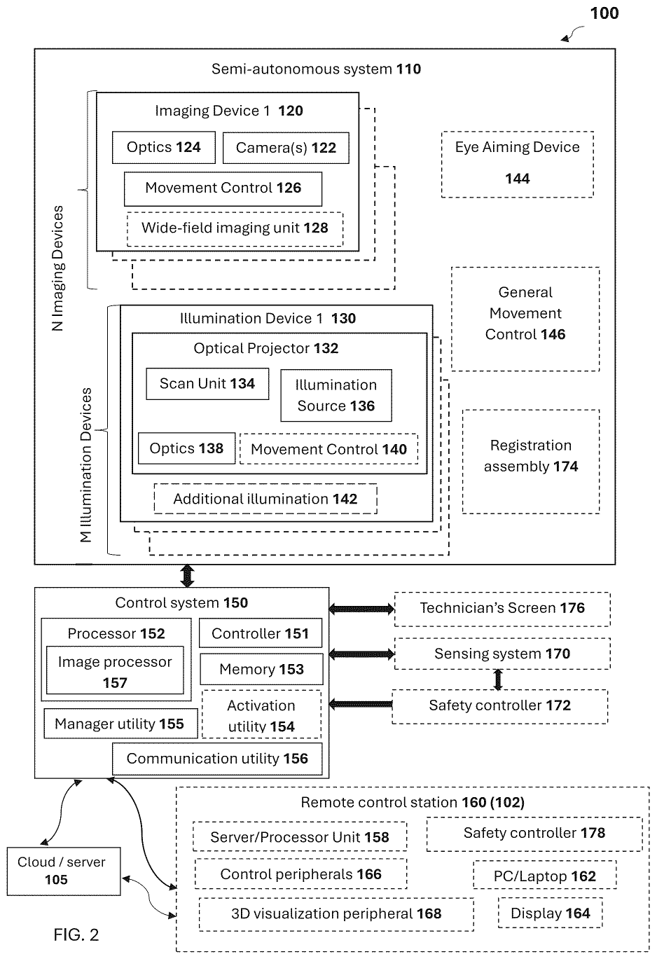

Reference is made to FIG. 2 showing by way of a block diagram the eye examination system 100 of the present disclosure. The system 100 includes, inter alia, a semi-autonomous system 110 and a control system 150. The system 100 is also configured for communication (using any known suitable communication technique) with at least one remote system at a remote-control station 160 which can serve, inter alia, as the eye physician's tool for operating remotely the semi-autonomous system 110.

As also exemplified in the figure, the system 100 and/or the remote-control station 160 is/are in data communication (using any known suitable communication technique) with the server system 105.

The system 100 may further include a technician's screen 176, a sensing system 170 and a safety controller 172. The technician screen 176 may be used to control the system, as well as system's parameters, similarly to the remote control system 160.

The remote control system 160 may include a data processor unit 158 which may also reside in an external server (remote control system).

The semi-autonomous system 110 is configured and operable to perform a number of examination sessions of the patient's eye and provide corresponding image data. The semi-autonomous system 110 includes at least one illumination device 130 and at least one imaging device 120. In some embodiments, the semi-autonomous system 110 may further include an eye aiming device 144, a general movement control 146 and a registration assembly 174.

The illumination device 130 includes at least one optical projector 132, each configured and controllably operable to automatically project a sequence of illumination spots of a predetermined shape on a plurality of locations extending in a spaced-apart relationship across an eye region during an eye examination session of the eye. The optical projector 132 includes a scan unit 134 (e.g., scanning mirrors) which is associated with an illumination source 136 and may also include various optics 138. In some embodiments, the projector 132 may include a movement controller 140.

In some embodiments, the projector 132 may include scanning mirrors mechanism (DMD), LCD, electronic DOE, etc., or a combination of them, which enables projections in various positions within its FOV without moving the illumination device 130.

As also shown in the figure, the system 110 further preferably includes an additional illumination unit 142, which may or may not be part of the illumination device 130. The additional illumination unit 142 provides backlight and or background illumination.

The optical projector 132 is configured and controllably operable to automatically project a sequence of illumination spots of a predetermined shape on a plurality of locations extending in a spaced-apart relationship across an eye region during an examination session of the eye. This sequence of illumination spots of a predetermined shape may be focused on a plurality of locations within the field of view of the optical projector 132. In some embodiments, the projector 132 may be configured to project radial patterns, concentric patterns, and multiple patterns (such as multi slit).

In some embodiments, the sequence of illumination spots may be projected in a time-apart manner, enabling specific exams, for example: pupil response to illumination, strabismus test, tears break up time, contact lenses dynamic movements etc.

In some embodiments, the optical projector 132 is configured and operable to automatically vary the spherical angle of projection of the sequence of the illumination spots with respect to the eye. The movement control 140 of the optical projector 132 may vary, automatically or manually, the angle of projection to any feasible angle relative to the field of view. The image data provided by the semi-autonomous system 110 thus includes image data pieces in association with data indicative of the orientation of the spherical angle of projection.

The illumination device 130 and the imaging device 120 may include, in some embodiments, optics supporting a depth of field that may be focused on an area that includes the entire anterior part of the eye (~13 mm). For implementing this depth of field, the illumination device and the imaging device may have each their own optics. However, in some embodiments, they may, partially or entirely, share the same optical path\axis.

The imaging device 120 of the semi-autonomous system 110 includes an imaging sensor 122, typically one or more cameras, associated with or including built-in optics 124.

The imaging device 120 is configured and operable to acquire focused images of the plurality of locations being illuminated by the illumination spots during the examination session and generate image data including at least the plurality of image data pieces in association with the respective plurality of locations. The image data is indicative of abnormalities in the location(s) within the eye region, enabling extraction of the abnormality-related information by further image processing and/or manually, by a physician. For example, the deformation of the slit may point to a pathology or a disorder in the eye structure. The physician examines the slit edges; therefore, they should be as sharp as possible at the focal point of both illumination and view parallel systems.

The same imaging device 120 or a separate imaging unit 128 is preferably further configured to operate together with the illumination device 130 or additional illumination unit 142 to implement a wide-field imaging mode.

The at least one imaging device 120 is operatable synchronously (using SW and/or HW synchronization) with the at least one optical projector 132 during the examination session to properly acquire the image(s) of the sequence(s) of illumination spots being projected.

It should be noted that, generally, the system 110 includes a number N (N≥1) of the optical projectors, and a number M (M≥1) of the imaging devices, where the numbers N and M being the same or different.

The image data can be processed by the control system 150 being integral with or connectable to the semi-autonomous system 110. The results of data analysis obtained by the control system, or raw image data provided by the imaging device 120, or both of such data, can be properly stored and duly communicated/forwarded to the remote control system at the physician-related station 160 and/or to the server.

The local operator or the physician can perform an asynchronous exam (“Store and Forward”) of the patient eye by performing a fully autonomous set of pre-defined exams and then scrolling between the generated images and reports of the findings of the examination session. This scenario of exam does not require simultaneous interaction with the patient/examination device. The medical information, including images and data obtained by the system 110 is stored and then forwarded to the physician/specialist who reviews and interprets the data. Alternatively, the physician can perform a synchronous (“Real-Time”) exam where he/she controls the exam parameters of the semi-autonomous system 110 and/or views the findings in real time. In this scenario, the exam is dynamically guided by the physician. For example, the physician may choose in real time the angle of a subsystem(s) of the imaging device 120, the angle of projection of the optical projector 132, the slit/projection type, width and intensity level, and then the semi-autonomous system 110 executes the chosen illumination and imaging parameters autonomously. Thus, the technique of the present disclosure provides an optimal approach for combining synchronous (real time) examination flows with asynchronous (“Store and Forward”) examination flows. The control system 150 is configured as a computerized system, having inter alia data input and output utilities (not shown), memory 153, processor 152. According to the technique of the present disclosure the control system includes a manager utility 155 and a controller 151. The latter may include or may be associated with an actuator utility 154 to actuate/trigger operation of the illumination and imaging devices as will be described further below. Also, the control system is properly equipped with a communication utility 156 for data communication with the remote physician-related station 160 and possibly also with the system 110 as the case may be. The control system 150 may also be associated with (may include or may be connectable to) a sensing system 170 and safety controller 172, as will be described further below.

The manager utility 155 is configured and operable to identify data indicative of an examination task in relation to the specific eye examination prescribed for a specific patient and define operational data for the semi-autonomous system 110. To this end, the manager utility may be responsive to input data including patient-related data and select the examination task data from data pre-stored in the memory in association with the respective operational data, or responsive to the input data also including the examination task data and select the operational data pre-stored in the memory. The operational data is used by the controller 151 to control the corresponding operation of the system 110. The operational data defines operational parameters of the examination session of the eye.

As will be described in more detail further below, the eye examination system 100 can function autonomously and is capable of conducting a series of examinations entirely by itself. A predefined set of examinations is stored e.g., in memory 153 of the control system 150. Each pre-defined examination with its respective pre-stored operational data is related to herein as a “preset” and is specific for each clinical exam (e.g., directed to detect/analyze specific pathology or eye part). Presets may be obtained/updated directly from the physician at station 160 (102 in FIG. 1), and/or from the server 105. The manager utility 155 is responsible for selecting the pre-stored operational data according to the specific examination task. For example, a pre-defined preset may include and is not limited to pre-defined values of illumination angle, imaging angle, slit shape and intensity, etc., required for a specific examination of cornea pathologies.

The presets can be predefined at the server and may be set by the system manager, physicians, as a set of operations for repeatable future examinations.

The technique of the present disclosure provides for optimizing management and performance of the examination procedure (image acquisition and image data evaluation modes). In this connection, it should be noted that in the world of telemedicine there are two main modes of operation-synchronous (real-time) and asynchronous (“Store and Forward”).

During the synchronous exam (real-time) procedure, the doctor controls the exam parameters of the device and views the findings in real time. The exam is dynamically guided by the doctor. Suitable examples may include that known as Davinci surgery robot, and also a device for remote eyeglasses fitting (subjective refraction).

During the asynchronous exam (“Store and Forward”) procedure, patient's medical information is collected and documented for later interpretation by a healthcare provider. This mode distinctively allows for both geographical and temporal separation between the doctor and the patient. Medical information, including images and data, is stored and then forwarded to a specialist who reviews and interprets it at a subsequent time, not requiring simultaneous interaction.

The eye examination system 100 of the present disclosure is capable of functioning autonomously or at least semi-autonomously to conduct a series of examinations entirely by itself. The system can track the examinee's eye and adjust accordingly, direct the examinee's eye towards the optimal orientation, execute autofocus tailored to the specific examination required, and autonomously and swiftly capture all images that may be necessary.

For example, as it is hard to focus on the cornea which in healthy person is transparent, the system may operate as follows: The system may focus the slit(s) projection on a high contrast element within the FOV (e.g., on the iris), and then modify the focal point to be at optimal position in order to bring the best focal point to the average distance from the iris, at the exam position and point. For example, the system may use the distance from the iris that was entered as data for a specific patient. Note that the average distance from the iris deduced from the general population may not be used for a myopic patient. In some embodiments, in order to detect the optimal focal point, the system uses the slit's projection (or multi slit projection) to analyze the curvature of the examined part and adjust the focal point location using the understanding of the examined object curvature.

Additionally, the control system 150 is capable of evaluating the quality of the images being acquired and allowing transition to the next examination, continuing this process until the entire series of tests is completed. Three classes of image quality may be defined, i.e., good, reasonable and poor, where images that were found of poor quality will require recapturing. Thus, for an asynchronous examination, the local operator can simply press a button to initiate the series of tests, which will then be sent for analysis by a physician. Alternatively, the physician can set the examination parameters and manage it synchronously in real-time, all while monitoring the examinee's eye. Alternatively, the system can be configured to acquire multiple sets of the same preset, and forwarding the best set, or forward a new set created using the best images taken from all the acquired sets, providing the same continuous coverage as a single set.

It should be noted that the image quality can be controlled using image processing heuristics, contrast level estimation, hue saturation measurements as known in the art or by using a dedicated Deep Neural Network for image quality estimation (for example CNN that is designed for classification of several quality classes. Moreover, in some embodiments the system ensures that all slit images in the set have been captured and that they provide complete coverage of the examined area using image processing methods, DNN models, etc. as known in the art.

Due to the fully or at least semi-autonomous examination capabilities, the technique of the present disclosure provides different control and automation levels, to be selected according to the required use-case. These include the following:
General Examination (asynchronous)—Fully autonomous execution of several predefined exams, each of them with its different preset. This can be initiated by personnel even without clinical training. These exams are conducted across different regions of the eye, enabling the eye physician to gain a broad overview of the eye's condition and to identify potential pathologies if present.
Selection of exam configuration (synchronous/asynchronous)—Selection from a list of exams, intended to address specific part of the eye or a suspected pathology. Upon selection, a set of several predefined exams, optimized to the selected eye part or pathology, is performed. This can be conducted with or without the eye physician's real-time observation.
Selection of exam parameters (synchronous)—in this mode, the examination is conducted while the eye-physician is observing the eye in real-time. This way the physician can get a “preview” of the expected acquisition. The physician can select various examination parameters such as: illumination and imaging angles, slit parameters—width, height, color, shape, zoom, aperture and intensity. The slit beam location can be controlled as well. Once satisfied with the preview, acquisition of series of images of the entire region of interest is performed.
Full manual control (synchronous)—“Micromanagement mode”, which is generally similar to other remotely controlled Slit Lamp solutions. This includes complete manual control of the examination device, including the various mechanical degrees of freedom and the slit positioning on the examined eye. This process is useful mostly for in-person examination scenarios.

One or more optional routes may include the following: the local operator is manually aiming the system in front of the eye, and then operates the system to perform the Auto Focus (AF) function (or adjust the focus manually), and angle and slit properties; and only then uses the automation of the image acquisition.

The system can operate in a so-called “hybrid exam mode” which combines the synchronous and asynchronous modes, as well as the above-described one or more optional routes.

The operation may for example be briefly as follows: When a new patient arrives at the exam location, the local operator initiates a default general exam series which is automatically acquired by the system 110. The acquired exam series is sent to the eye physician at station 160. The eye physician reviews the exam series, while dynamically positioning the slit in each exam. If the doctor locates or suspects a finding and wants to further examine the suspected area/pathology, the doctor can view the eye in real time while obtaining a relevant exam series, for example, corneal exam series. In extreme cases, when the relevant exam series does not provide a satisfactory answer, the doctor can request an exam with a specific parameter set. For in-person examination, the manual mode can also be utilized.

It should be noted that the system 110 of the present disclosure is configured to support doctor viewing modes. The system is designed to provide a sense of continuity, and be intuitive for the doctor, similar to the traditional Slit Lamp examination. As such, the transition between the acquisition process and viewing the acquisition results is intuitive and smooth. Switching between viewing exams at different parameters is also intuitive. For instance, while switching to another exam, with a wider slit, the slit initial position will remain wherever it was left in the previous exam, so the doctor's experience is just like widening the slit during the traditional exam.

In addition, the acquisition results can be viewed through multiple user-friendly modes including video mode, slit position change mode, and frame-by-frame transition mode. The video mode provides automatic and sequential transition among the multiple slit frames, the slit position in each frame is slightly different from the last one, collectively covering the region of interest (might be the entire anterior segment of the eye). The doctor gets the impression of a video of the slit transition across the eye. The doctor can control the “video speed” whenever required. The slit position change mode provides that the doctor can conduct a controlled slit-scan of the eye using a slider. With this slider, the slit can also be “aimed” on the desired area of the examined eye. The frame-by-frame transition mode provides that a frame with the desired location of the slit can be selected from a gallery. This mode is useful whenever a finding was located and the doctor is interested to observe it with slightly different slit locations, The above modes can be used interchangeably, to allow efficient video viewing while on the other hand, allowing exam quality as at any given moment, the doctor can linger on a certain location, in order to think, tag the finding and digitally zoom on the desired area. The system also provides more advanced viewing modes for documentation, comparison to previous exams and referral for second opinion purposes.

The above and other details of the technique of the present disclosure are described in more details further below.

The illumination and imaging devices may generally have any known suitable configuration. The illumination source 136 may be one of the following or a combination of them: Halogen/Xenon, LED(s), Laser, any light/energy source known in the art. The illumination source 136 may use narrow or wide spectrum light. The color of the illumination may be monochrome or combination of monochrome and wide range color. The color may be outside of visible spectrum, i.e., infra-red or combination of several wavelengths. The illumination source 136 may include band-pass or cut-off optical filters. In some embodiments, the illumination source may include a DMD, LCD. Electric DOE, and other subsystems that may produce the properties of the spot as a projection.

The optics 138 may include one or more lenses for obtaining the required illuminated region of interest (ROI) on the examined eye and/or one or more apertures for obtaining the required depth of field (DOF). The aperture size may be constant or variable by manual or software control. The change of aperture size may be triggered by another system component or manually. The aperture size can be configured in accordance with the predefined pre-set or manually as requested by the physician. The optics 138 may additionally include polarizers and/or filters.

The scan unit (illumination projection means) 134 may be configured using one or more of the following techniques: programmable LCD or DLP projection, micro-LED projection, use of electric Diffractive Optic Elements (DOEs), hardware predefined patterns and switching among several such patterns.

In general, the illumination device 130 may include motors, rails, encoders, limit sensors and any other components for radial and linear movement and their control may be performed by the movement controller 140. The mechanics may include any combination of any of the possible six degrees of freedom and any amount of the degrees of freedom may be motorized. Additionally, the optical projector 132 may include a focusing mechanism, in particular, it may include autofocus support.

In the embodiments where a laser is used as the illumination source, the optical projector 132 may include defocusing option to define specific depth of focus. In such embodiments the optical projector 132 may also include a distance sensor.

Additionally, in some embodiments, the relative angle between the cameras (stereo angle) can be modified manually or automatically.

It should be noted that the semi-autonomous system 110 of the present disclosure resolves a major problem of distorted images encountered during traditional slit-lamp examinations. In a typical traditional slit lamp examination, at every moment of the examination, the eye-physician sees sharply and in high-quality, only fragments of clear image, while the other parts are blurred, distorted, not in focus, or even unseen. The eye-physician is aiming and focusing multiple times on various parts of the eye, gathering all the observed information to be reconstructed to a complete three-dimensional model of the examined eye in his mind. Due to a very limited DOF of the microscope and the illumination of the slit lamps known in the prior art, the eye-physician must or should scan through the various layers of the eye, residing on different distances from the microscope and the source of light, in order not to miss pathological findings. These limitations are still present in the existing prior art of “Manually Remotely Controlled Slit-Lamps”. However, when observing a set of images on the screen, the physician is unable to compensate in his mind for all those distortions, as a bad image remains a bad image.

The semi-autonomous system 110 of the present disclosure resolves the above mentioned problem by providing optical systems with extended DOF for the sensor of the imaging device 120 (as will be described further below) and the illumination device 130, in addition to autofocus support. This provides a clear and sharp slit view of all the observed layers of the eye on the same image. The DOF of the optical projector 132 is wide enough to include multiple anterior segment parts of the eye and the adnexa at once in focus. As will be described below, the short acquisition duration allows the capturing of all the required images prior to any major eye movement, in most cases.

The autofocus operation of both the illumination and imaging devices may use standard and well known in the art image system planners. For example, during automatic operation of the semi-autonomous system 110 of the present disclosure, standard Focus Measuring Function (FMF) may be used to measure the relative focus quality and a standard stochastic search may be applied in order to detect the global maximum of the image sharpness. Additionally, the illumination auto-focus adjustment may use the imaging device in order to adjust the illumination focal point using the standard FMF methods examining the projections at the desired point(s).

As already mentioned above, the illumination device 130 may include additional illumination unit 142 (or the illumination device 130 may operate with additional illumination mode) and operate together with the imaging deice 120 (e.g., additional imaging unit 128) to obtain a wide-field image of the eye. This enables to align foreground image formed by the image data pieces related to the sequence of illumination spots on a background image formed by the wide-field image of the eye, as will be described in detail further below.

The additional illumination 142 may include a direct or indirect illumination source and the illumination intensity and/or color may be controlled. The control may be performed by software, triggered by another system component or manually.

In some preferred embodiments of the present disclosure, the optical projector 132 is configured and controllably operable to automatically project a sequence of slit-like shapes on a pre-defined plurality of locations extending in a spaced-apart relationship across an eye region during the examination session. In other embodiments, each projected illumination spot may have a predetermined pattern suitable for diagnosis of different parts of the eye and the ocular adnexa.

Furthermore, in some embodiments, each illumination spot may have a predetermined pattern suitable for diagnosis of most of the parts of the eye and the ocular adnexa (which enables shorter examination duration).

The projected pattern can be either single pattern projection or a series of projected patterns of the same kind, or shifted patterns that form the same type. The aggregation of the shifted series of adjacent patterns might provide coverage of the entire region of interest. The single pattern may have the shape of a vertical line/slit, similar to the shape/pattern used in conventional slit-lamps. The lines may vary in their direction, i.e., horizontal, diagonal, etc.

The optical projector 132 may be configured to project slits having a general shape, e.g., concentric, radial or any other general form. The projected patterns/slits may be projected in various widths (per slit) and heights, with any number of slits/patterns and with various constant spaces between the slits. The projected patterns may have variable spaces between the slits. The slits in different patterns can be partially or completely overlapping.

In some embodiments, described in more detail further below, projection of the sequence of illumination spots includes projection(s) of a multi-slit shape/pattern, i.e., an array of spaced-apart slit-shaped illumination spots. Typically, two or more such multi-slit patterns are sequentially projected on the eye region. The optical projector 132 is configured and controllably operable during the examination session to automatically project the sequence of multi-slit patterns on the eye region such that the illumination spots are focused on a plurality of locations extending in a spaced-apart relationship across the eye region.

Keeping in mind the focused slit-width limitation and that of the space between the slits, and the desired scan of the entire eye region during the examination session with minimal duration of the session, the multi-slit projection mode is preferably implemented in an interlaced fashion. First and second sets of the illumination spots are successively (in timely separated fashion) projected onto, respective, first and second sets/arrays of spaced-apart locations, wherein the first and second locations are arranged in the interlaced fashion. The first and second locations may be partially overlapping.

It should be noted that the interlaced mode is preferably used for retinal imaging. As for imaging of the anterior part of the eye, a so-called “semi interlaced” mode may be used, where all the anterior part is covered with nonoverlapping slit/multi-slit series being projected (partial overlap between the slits/multi-slits can be used in order to provide semi continuous slit scan).

The camera(s) utilized by the imaging device 120 may be monochrome, color, multicolor, IR or any other wavelength or combination of several (including being operable simultaneously) wavelengths. Instead of moving to a different capturing position, multiple cameras located at different positions may be used. In some embodiments, each camera may have different optics. Moreover, in some embodiments one or more cameras are integrated in order to extend the depth of field or to enable acquisition of the anterior and posterior parts of the eye simultaneously. Similarly, in some embodiments, multiple projectors, located at different positions, may be used, and may have different optics.

In some embodiments, stereo acquisition by two cameras may be used to acquire focused images of the plurality of locations being illuminated by the illumination spots during the examination session. Any pair of cameras may be used for capturing a stereo image of the eye region for purposes of three-dimensional visualization to the eye-physician (similar to the traditional binocular slit-lamp examination).

In some embodiments, multiple cameras may be used to obtain higher resolution and/or more data and/or more accurate measurements.

It should be noted that in some embodiments, multi-lens imaging devices can be used in order to capture the 3D shape of the illuminated spots.

Generally, the technique of the present disclosure can utilize any known 3D imaging technique and is not limited to stereovision or multiple vision.

Each camera of the imaging device 120 may have an extended DOF, in the range of 10-14 mm, and/or may have an automatic focal setting.

It should be noted that the DOF of the optical projector 132 and the DOF of the imaging device 120 are independent and may be independently controlled (e.g., by the controller 151 of the control system 150). It should, however, be noted that the DOF enables the projection and the imaging to be concentric and therefore enables to achieve an extended DOF of the system without limiting the respective DOFs of the illumination and imaging devices to move together.

More specifically, when using side projection (i.e., projecting from an angle with respect to central axis of the eye), the high depth of field enables the illumination (and correspondingly the imaging) to be in focus at all illuminated (captured) positions.

The extended depth of field enables to project and capture the entire slits' set without using the motors or moving parts in the autonomous acquisition session, since it enables capturing all/most of the anterior part of the eye in the same view.

In some embodiments, a trigger mechanism may be used between the illumination source 136 and the camera(s) 122 to ensure any one of the following: simultaneous illumination and acquisition for multiple imaging devices, transition between consecutive patterns in the pattern sequence, and shortening of the required time for the image acquisition process.

More specifically, transition between consecutive patterns in the pattern sequence may be as follows: A pattern is projected; a trigger is sent to the imaging device (camera) to capture the respective image; upon completion of the image acquisition, the projector is triggered to project a second (next) image, and when the next pattern is projected, the camera is again triggered, and so on, until all the predefined patterns of the specific preset are properly projected and imaged. The images are kept stored (e.g., the system may store the images locally, e.g., in the camera memory) until the preset patterns are projected and imaged, i.e., until the required number of image acquisitions of the examination sessions is performed; and then the images are forwarded (e.g., send to the backend) for further processing or viewing.

In some embodiments, an eye/pupil tracking mechanism may be implemented using the above-described imaging device utilizing imaging sensors involved in the eye examination, e.g., camera(s) 122, or by using an additional sensor for this purpose, or by implementing other eye-tracking solution (e.g., using second dedicated camera, or using IR illumination combined with another camera (with VIS cut filter, while the examination system uses IR cut filter), etc.). The eye/pupil tracking may be operated automatically by the control system 150 (e.g., controller 151) during the examination session.

In some embodiments, the eye tracking may include iris tracking. Eye tracking, Iris tracking or pupil tracking can be performed using any known suitable technique(s), e.g., known in the field of image processing and deep learning, utilizing RT segmentation mechanism, as well as any known suitable RT eye tracking methods. Such technique(s) advantageously provide(s) for positioning the system in front of the eye; allows automatically centering the system; as well as image stabilization and slit projection stabilization. Each of these advantageous features improves image quality and/or images set quality, and/or the entire exam clinical quality.

The optics 124 of the imaging device 120 may include one or more of the following: lenses, apertures, polarizers, filters, and other optical elements. Specifically, lenses may be used for imaging the required region of interest (ROI) of the examined eye, a limiting aperture may be used for obtaining the required DOF. The required DOF may be wide enough (10-14 mm) to include all the anterior segment parts of the eye and the adnexa at once in focus. In addition to the aperture size, the resulting DOF also depends on the specifications of the lens being used. The aperture may be of a constant size or of a variable size (manually variable or electronically variable (being controlled by suitable software). The optics 124 may provide optical magnification capabilities. This functionality may be controlled manually, or electronically (using pre-programmed software, e.g., in response to activation by a trigger signal from another component).

The optics 124 may further include internal moving parts or other mechanisms allowing focusing on various depths/structures/layers of the examined eye. In particular, the optical design may enable capturing of both the anterior and posterior (retina, vitreous, choroid and the optic disc) segments of the eye.

The movement controller 126 of the imaging device 120 may include some or all of the following components: motors, rails, encoders, limit sensors and any other components for radial and linear movement and their control. Any combination of any of the possible six “Degrees of Freedom” of movement may be controlled. In particular, the distances between the camera(s) and the eyes, and/or the angles between the optical axis(es) of the camera(s) and the eye surface may be adjustable and the mechanical and electronical elements controlling these distances/angles may be motorized and/or computer controlled. The movement controller 126 may further include such functionalities as auto-zooming and autofocusing (for example including a distance sensor). Generally, auto focus is a programable mechanism that can be performed using dedicated HW, or a combination of HW & SW.

As mentioned above, the imaging device 120 of the semi-autonomous system 110 may include the wide-field imaging unit 128 configured to obtain a wide-field image of the eye, as exemplified in relation to the additional illumination 142. In some embodiments, a dedicated wide-field imaging unit 128 may not be required and the background wide-field image of the eye may be captured by any one of the cameras of the sensor 122. Similarly, a dedicated illumination unit 142 might not be used but the same illumination source 136 can be used while in an inoperative state of the projector device. A zoom lens (electronic) may be used for this purpose (wide range imaging).

As stated above, in some embodiments, the semi-autonomous system 110 includes an eye aiming device 144 (generally termed “fixation target”). In some embodiments, the fixation target can be accommodated in the imaging axis using a beam splitter/combiner.

The fixation target is synchronized and set relative to the current pre-set and exam needs. This can be done automatically, e.g., using vocal instructions to the user to track it.

The fixation target is used for aligning the examined and/or the second eye to a desired direction or position (i.e., might replace part of the motor movements). The fixation target/eye aiming device 144 may be used for stabilizing the image of the eye by reducing the frequency and/or amplitude of eye movements and for returning to the desired position after such a movement. The eye aiming device 144 may be implemented by a display or LED or laser marker or any other aiming solution and may be motorized (e.g., in case of aiming by a LED). Additionally, the eye aiming device 144 may be controlled and operated automatically by the controller 151 such that the LED/pixel/target on the screen is turned on according to the direction required by the examination task being performed.

It should be noted that the illumination and imaging assemblies/components are configured to enable separate movement thereof, while in some cases or exams they need to move together. It should also be noted that in the embodiments where separate movements of the various components, belonging to either illumination device 130 or imaging device 120 are used, bigger parts of the system, for example, all the components of illumination device 130 and imaging device 120 may be moved together (having any linear or angular degrees of freedom) to place the semi-autonomous system in the desired position and angular orientation in front of the examined eye (and for switching between eyes). This “large scale” movement of the whole system may be controlled by the general movement controller 146. It should be noted that such movement is generally performed once, before the beginning of the examination session and does not affect the fast image acquisition during the examination session itself.

As described above, the control system 150 includes manager utility 155, controller 151, processor 152, memory 153, communication utility 156, and in some embodiments, activation utility 154. The manager utility 155 responds to input data, obtained from a local operator's control unit (e.g., technician's screen 176) or from the remote control station 160 (e.g., PC/Laptop 162) to select a set of one or more presets (predefined exams), optimized for the selected eye part or pathology indicated in the input data and including respective operational data. This operational data defines all parameters that may be necessary that are required to capture all images that may be necessary for each of the one or more presets (predefined exams).

The controller 151 is configured to operate the semi-autonomous system 110 in accordance with the operational data provided by the manager 155. This includes, but is not limited to, operation of the optical projector 132 in a prescribed mode (for the specific examination session), i.e., single-slit and/or multi-slit projection, angle of projection, slit dimensions, etc., and to operate the imaging device 120 synchronously with the optical projector 132. The operational data on the one hand defines the optimal examination parameters for the specific examination task to enable the physician to obtain and analyze maximum required information in the optimal format, and, on the other hand defines optimal examination conditions such that a duration of the examination session can be desirable short (e.g., for example shorter than a periodicity of large saccadic movements of the eye), thereby minimizing image deterioration factor in the image data and minimizing time of the eye exposure to the illumination during the examination session.

The controller 151 is configured as a computing device, being any one of the following: a microcontroller, a desktop computer, a laptop, a small or mini-computer, single board computer, DSP, etc.

The controller 151 is configured and operable for managing operation (commanding) of all the peripherals including illumination device 130, imaging device 120, alignment mechanism (including eye aiming device 144 and general movement control 146), etc. The controller 151 manages, via control signals or commands the illumination pattern projection and image or image sequence acquisition, and motion control of all degrees of freedom by sending motion control signal (motion commands) to bring respective elements (movable parts) to the desired positions while monitoring the actual position as reported by driver cards controlling the motors directly. The controller 151 is in charge of performing higher-level tasks such as auto-aiming, autofocus, auto-acquisition, testing image sequence's validity, grading, quality and its approval or rejection, managing the acquisition of sets of exams, and possibly also performing image processing procedures, etc.

In particular, as part of the automatic operation of the semi-autonomous system of the present disclosure, the controller 151 is in charge to perform ocular repeated capture with different illumination, photography and other system parameters which may be initiated by the remote eye-physician or the local operator out of predetermined examination protocols for each pathology/disease stage/or part of the eye.

As already mentioned above, the controller 151 is in charge of communicating with the server/processor unit 158, via the communication utility 156, via an internet connection or via any other interface with other components or devices of system 100. The controller 151 drives one or more of the following: moving parts, motors, controllers, drivers, slides, gears. The controller 151 may support automatic or semi-automatic movements of the optical projector 132 and camera sub-systems of imaging device 120 towards the eye. The controller 151 may also monitor the position of all the motors of all degrees of freedom, as well as their homing, switches, optocouplers, optical sensors (e.g., optical sensors that react as microswitches), and any other hardware limits.

The processor 152 may be responsible for running a part or all the image processing algorithms and any other required algorithms and software for the operation of the semi-autonomous system 110. In particular, the processor 152 may include an image processor utility 157 configured and operable to analyze the image data generated by the imaging device 130 and generate data indicative of the number of abnormalities in the respective number of illuminated locations in the eye.

The server/processor unit 158 at the remote station 160 may be configured to perform one or more of the following tasks: image processing, including image reconstruction, examination matching between eye-physician and patients, managing medical data/files of patients, including history and previous examinations, and other medical and general tasks as done in modern systems.

More specifically, the image processing tasks, that are typically performed off-line, and typically are performed on the cloud, but may generally be performed by the control system 150 or the tasks may be distributed between the processors 152 and 158, may include and are not limited to:
(i) Cutting the slits from “multi-slit” images and reconstructing the image, such as pasting the cut slits on top of a background image, thus, generating multiple single-slit images;
(ii) Identifying the different parts of the eye, such as sclera, adnexa, pupil and iris (this is performed either by cloud computing or at the physical unit 110 (in order to perform autonomous centering in front of the patient eye);
(iii) Identifying and removing the specular back-reflections;
(iv) Ordering slits from different sets (single slit sets); or constructing an ordered sequence of frames or a video from the reconstructed images;
(v) Generating a three-dimensional partial or complete model of the eye for imaging capabilities (this may be AI-based modeling procedure).

As mentioned above and will be described further below, the technique of the present disclosure provides optimal data (image data) enabling use of AI-based data processing, in particular suggestive AI modeling procedure, for data analysis which can also be used for optimizing the examination procedure.

It should be noted that the construction of the ordered sequence of frames of video is implemented when utilizing multi-slit imaging. Considering ordering of slit from different single-slit sets, this may be wide slit sets, medium slit sets, or thin slit set. The system is configured and operable to enable the physician to move from wide to thin slit on the same location (e.g., by rolling the mouse roller back and forth). To this end, the slits are to be in a specific order. For example, when utilizing a wide slit, a set of two medium slits and 5 thin slits are used on the same location, which requires data indicative of the position of each one of these slits in its set. It should be noted that the technique of the present disclosure allows for imaging wide and thin slits being projected and rendering the medium size slits.

Regarding the generation of the 3D partial or complete model of the eye, it should be noted that the 3D model of the eye may hold information such as geometric information (due to 3D calibration), and this information can be used as aid to crop single slit from a multi-slit image, etc. The 3D model enables conversion of pixel to inch/mm scale.

In some embodiments, the server/processor unit 158 may be responsible for storing of one or more of: images (and their meta-data of acquisition parameters), medical data, entire medical files for the purpose of visual documentation, follow-ups, sending the examination data to second opinion etc.

In some other embodiments, the processor unit 158 may be in charge of managing the flow of pairing examination units with physician units, and/or the examination flow, monitoring the system usage, and enforcing an encryption and data security protocols.

The technician's screen 176, which may function as a local operator's control unit, may include one or more of the following control accessories and functionalities (not indicated in FIG. 2): control accessories, e.g., keyboard and/or a joystick and/or a mouse/ or a touch screen; control accessories allowing the local technician to operate the examination device; control accessories assisting the remote eye-physician to conduct the examination; user interface offering options for initiation of predefined or manually selected sets of examinations. The technician's screen/operator's control unit 176 may be simply a screen and/or other display, may be a touch screen and/or be adjustable, allowing rotation of the screen for more convenience. The display may allow the local operator to review the acquired images and decide whether recapturing is required.

As mentioned above, in some embodiments, the system 100 includes a registration assembly 174, a sensing system 170 and a safety controller 172.

The registration assembly 174 is used for registering a position of user's face during the examination session and may include a chinrest mechanism or a face cradle for fixation of user's face at the registered position during the examination session. The registration assembly 174 may include a support platform carrying a face cradle defining a face support surface for supporting the user's face at the registered position during the examination session such that user's eyes look towards the eye aiming target 144. The face cradle may include or may be without a chinrest element.

The sensing system 170 is configured and operable to monitor user's face position with respect to the face cradle and generate corresponding sensing data to be analyzed for selective generation of a control signal to enable or disable the operation of the system 110 (i.e. the examination procedure). The sensing system 170 may include one or more sensors on the face cradle for monitoring the degree of contact of the user's face to the face support surface. Such one or more sensors on the face cradle may include at least one of the following: at least one pressure sensor, proximity sensor, or at least one IR sensor. Generally, one or more pressure sensors may be used to monitor the contact of the user's face with the face support surface; alternatively, or additionally an imaging device can be used.

The safety controller 172 is responsive to the sensing data and is configured and operable to analyze the sensing data and generate the control signal to the controller 151 upon identifying the safety condition of the user's face. The activation utility 154 of the control system is configured and operable to activate the semi-autonomous system to perform the examination session in response to a control signal indicative of a safety condition of the user's face being at the registered position. When the sensing data analysis is indicative of that the alignment of the semi-autonomous system with respect to user's face does not satisfy a predetermined requirement and may correspond to a predetermined risk condition, the safety controller 172 notifies the controller 151, which in turn sends, through the activation utility 154, appropriate commands to the various system components, e.g., to halt movement of specific devices.

The remote-control station 160 may include, inter alia, the processor unit 158, a PC/Laptop 162, a display 164, control peripherals 166, 3D visualization peripheral 168, and a safety controller 178. The PC/Laptop 162 may be part of the system 100 or may be a 3rd party's PC/Laptop (generally, any device, such as Laptop\PC\Tablet\smartphone, with communication capabilities and display can be used). In general, PC/Laptop 162 serves as the eye-physician's tool for remotely operating the semi-autonomous system 110 but may be used for operating the system 110 locally as well. Some of the additional functions that the PC/Laptop 162 may perform include: allowing the eye-physician's to observe the original or reconstructed images by scrolling through the single slit and/or multi-slit images (or any other relevant data in case of usage as imaging device); serving as the eye-physician's tool for viewing other clinical data of the patient; performing the diagnosis (e.g., using marking findings, writing remarks, clinical and non-clinical, scanning history, etc.); making decisions regarding the next steps; serving as the eye-physician's tool for “ordering” additional acquisitions of the patient's eye with selected parameters.

The display 164 may be a conventional 2D display or a standard 3D display screen implemented by, e.g., a VR headset or other three-dimensional solutions (denoted as 3D visualization peripheral 168 in FIG. 1) such as holograms, technologies of image projection to both eyes, etc.

In some embodiments, the sensing system 170 is configured and operable to communicate the sensing data to the safety controller 178 at the remote control station 160, where the sensing data is analyzed, and the control signal is selectively generated and communicated to the controller 151.

Additional parts of system 100 not shown in FIG. 2 may include one or more of the following: a chin and forehead-rest, an armrest, exchangeable optical sub-units for different examinations of different eye segments or diseases, additional optical elements such as folding mirrors and prisms. Possible external add-ons may include, but not limited to Gonio lens, external mirrors, contact lenses.

The system 100 may further include means for manual or software controlled change of the optical systems parameters, such as changing the distances between the different lenses inside the optical system or replacement, addition or removal part or all the lenses.

The system 100 can be used as a standalone system or a subsystem of another system.

As described above with reference to FIGS. 1 and 2, the system 100 can be in data communication with the cloud computing system 105 which implements the heavy data processing tasks. The cloud computing can be either on actual cloud service, remote PC, or even in local PC of system 100.

It should be noted that the technique of the present disclosure can operate with/utilize AI-based processing of the image data to identify suspected areas/parts and observations, to detect abnormalities/pathologies of the eye. Indeed, since the system of the present disclosure utilizes autonomous or semi-autonomous eye examination system configured as described above, the image data collected by such system includes high-quality images of various eye regions of various patients being collected by the same system (the same operational conditions), and moreover, such image data is less affected or even unaffected by errors induced by different operators. This provides optimized data for machine learning processing, in particular suggestive AI based processing. In particular, the suggestive AI-based technique can further be used for automatically optimizing the operational data according to the updated findings.

Typically, AI-based functionally can be installed in a remote control system (system 105 in FIGS. 1 and 2) being a server system (a computerized system including input and output utilities, memory, processor, etc.), connectable, via communication network, to subscriber eye examination systems of the type performing semi-autonomous eye examination procedures. Each subscriber system has its unique ID properly assigned to such system in a subscription procedure. The subscriber system sends to the server the image data obtained by the system in association with the preset data and/or examination task data being used to obtain the respective image data, and possibly also other relevant patient-related data. The patient-related data may include patient's IP and the server may access (at the server or at the physician-related station as the case may) the other patient-related data such as historical data of previous eye examination results of the patient. The server system is configured and operable to perform model-based processing (AI-based processing) of the input data received from each subscriber system and generate output data. Such output data may include data indicative of suspected parts and observations in the eye region being examined to enable detection of abnormalities and/or pathologies; and/or data indicative of detected abnormalities and/or pathologies in one or more parts of the eye region being examined; and/or quality level of the image data provided by the subscriber system; and/or data indicative of recommended preset data for examination session(s) to be performed by the specific subscriber system for eye examination.

Such recommended preset data may include optimized operational parameter(s) of the system, e.g., pattern(s) to be used, which is optimized with respect to specific part(s) of the eye and/or specific abnormality to be detected, and/or with respect to specific patient. For example, the AI-based processing may identify not sufficient image quality and generate recommendation data (e.g., optimized preset data) indicative of optimized operational data to improve the image quality. The system 100 (its manager utility) may be responsive to such optimized/updated preset data to initiate automatic performance of additional examination session using the optimized operational data. Additionally or alternatively, the AI-based processing at the server 105, based on analysis of historical data about previous eye examinations of the specific patient, may generate optimized preset data with respect to a specific examination session for the specific patient, and communicate respective data to the examination system 100.

In some embodiments, specifically, the AI subsystem is capable of performing one or more of the following functions: detecting poor-quality images and notifying the user (via user interface of system 100 and possibly also at the physician system, as the case may be) about the issue, recapturing poor-quality images (as the acquisition and projection parameters are retained within the system), detecting suspicious areas/parts, observations, or pathologies and marking them for the user, autonomously offering or performing additional acquisition sets using the existing presets or the AI generated presets that are suitable for further investigation, and potentially concluding its findings, if any. It is optional that the user or operator retains the capability to manually override the AI's decisions.

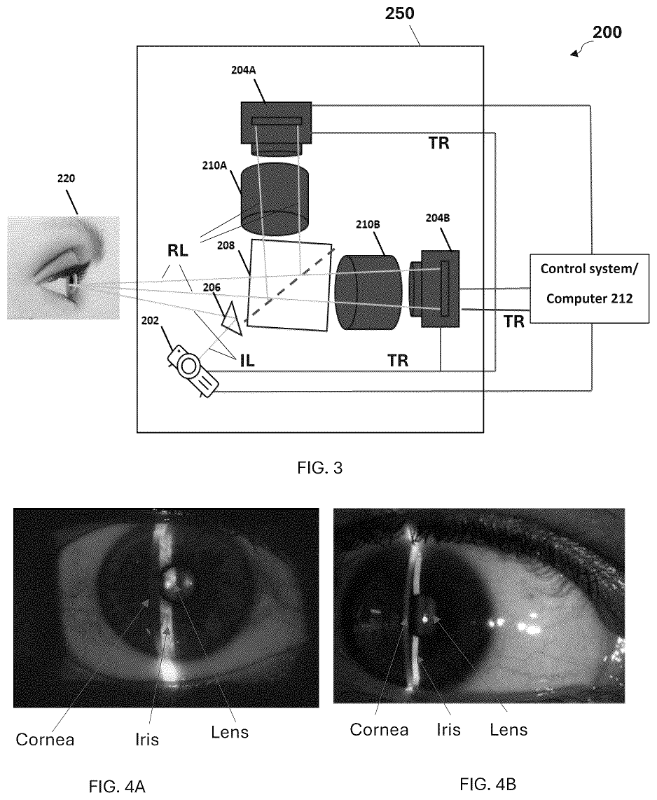

FIG. 3 exemplifies an eye examination system 200 of the present disclosure including a semi-autonomous system 250 (presenting a non-limiting example of the system 110 described above) and control system/computer 212 (which may constitute the control system 150 or control system 160). In the present non-limiting example, the semi-autonomous system 250 includes two digital cameras 204A and 204B for stereo acquisition of the plurality of locations of the eye 220 being illuminated by the optical projector 202 during the examination session. The use of a pair of cameras allows three-dimensional visualization to the eye-physician (similar to the traditional binocular slit-lamp examination). It should, however, be understood that the technique of the presently disclosed subject matter utilizes autonomous or semi-autonomous examination system which can be configured for mono or stereo vision, and the principles of the technique of the present disclosure are not limited to the use of 3D imaging.

Also, in the present non-limiting example, a beam splitting/combining mechanism is used to implement 3D imaging. However, it should be noted that the implementation of the technique of the present disclosure is not limited to this configuration. For example, two direct imaging axes (without using beam splitter) can be used with e.g., micro-lens technology.

The optical projector 202 includes an illumination source, illumination projection means (e.g., scanning mirrors) and various optics.

Illumination IL is coupled/incident onto the eye 220 at a predetermined angle using a mirror/prism 206. Light, RL, reflected from the eye 220 is collected (typically at a normal direction with respect to the face of the patient). The reflected light RL is split into two propagation paths by a beam splitter 208 and directed into the two digital cameras 204A and 204B. The digital cameras include respective lens modules 210A and 210B which may include several lenses and an autofocus mechanism to obtain focused images with an extended DOF.

It should be noted that the optical axis of the eye 220 may have various directions with respect to the optical axis of e.g., the digital camera 204B which may be defined by the eye aiming device 144 of FIG. 2 and is not shown in FIG. 3.

The operation of the system 200 is managed and controlled by the control system 212 which is configured and operable as described above in relation to control system 150 of system 110 being responsible to send trigger signals TRs to the optical projector 202 and the two digital cameras 204A and 204B such that the projection of the illumination spots and the acquisition of the sequence of the respective images are performed synchronously and are preferably configured to be fast enough to eliminate image blurring due to eye movements, such as saccadic eye movements, blinking, eye movements (due to for example patient eye fatigue), etc. As described above in detail with reference to FIG. 2, the control system 212 controls the operation of the optical projector 202, the digital cameras 204A and 204B, and is configured and operable to utilize data indicative of an examination task to select pre-stored operational data for the semi-autonomous system 250. The operational data defines operational parameters of the examination session of the eye.

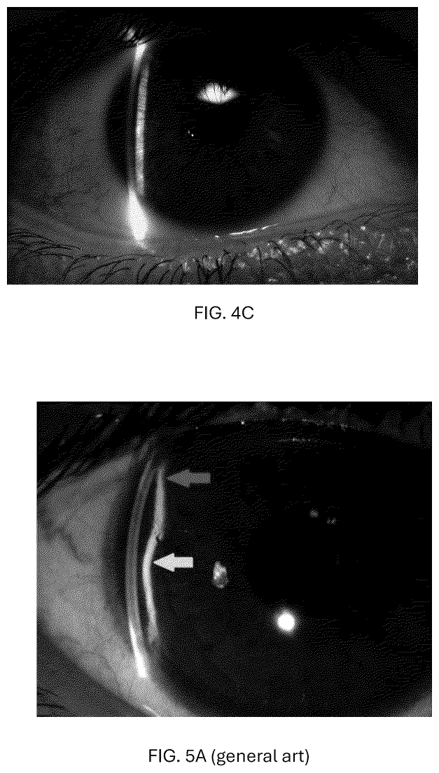

FIGS. 4A to 4C show examples of single-slit images obtained using the eye examination system of the present disclosure. FIG. 4A shows illumination using white color; FIG. 4B shows illumination with high intensities of Blue channel; and FIG. 4C demonstrates the need in different illumination intensities for examining different parts/regions of the eye It should be noted that in images of FIGS. 4A and 4B (using such different illuminations), both facets of the eye lens (frontal and rear) can be observed. FIG. 4C shows that although the illumination intensity is sufficient to properly observe the slit on the cornea and on the iris, there is saturation above and below the iris (on the sclera) requiring lower illumination intensity if sclera is to be examined as well.

It can be seen that sharp images of the slit can be obtained simultaneously for reflections from the cornea, the iris and the lens posterior capsule, which confirms that a DOF of about 12 mm can be obtained with the eye examination system of the present disclosure.

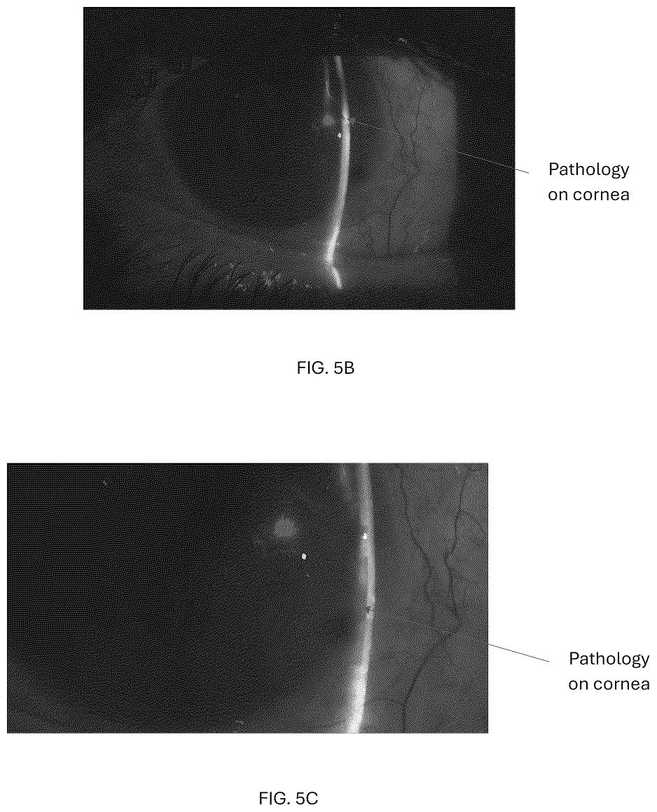

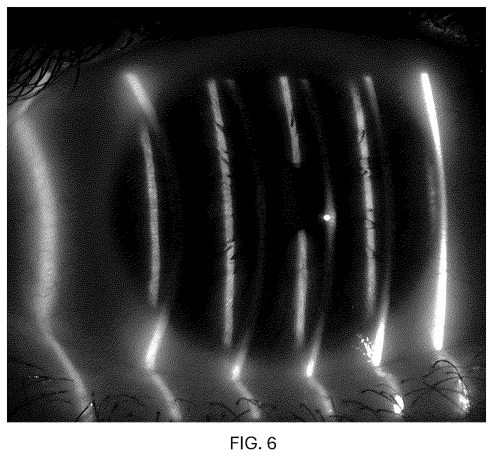

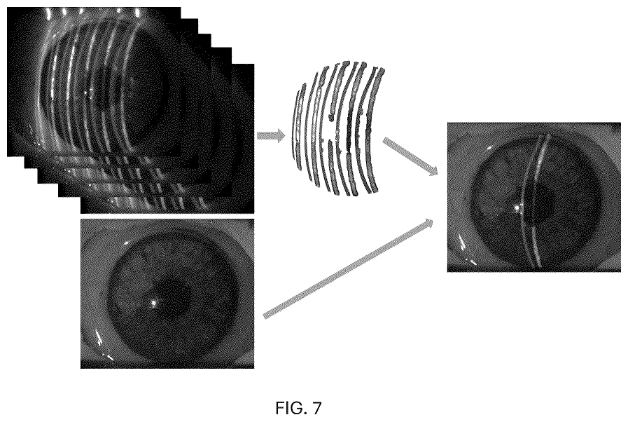

FIG. 5A shows an example of a single-slit image with marked pathological findings. In healthy eye, it is expected that the projection on the iris is relatively uniform. However, as can be seen in this example, the slit's projection on the iris has deformation in shape, width and color. As noted above, the deformation of the slit may be associated with a pathology or a disorder in the eye structure.

FIGS. 5B and 5C show examples of single-slit images enabling to extract pathology related data. In the example of FIG. 5B, a post trauma eye is shown with corneal scar. The width of the projected slit on the cornea presents varying thickness and a point where the inner layer (Descemet layer) is torn (where the arrow points at). FIG. 5C shows the other location of the same eye, the arrow points at the area where the cornea has varying thickness.

FIG. 6 is an example of a multi-slit projection on a real eye. The images of the slits can be seen on the cornea, iris and in the lens behind the pupil.

Image reconstruction is utilized in embodiments where multi-slit patterns are projected on the eye. This technique utilizes a so-called “image slicing” which relates to image reconstruction where a part of the acquired image is extracted and pasted on another image.

FIG. 7 shows an example of such image reconstruction where multiple multi-slit images were acquired, possibly with a predetermined shift in the relative locations of the slits in the neighboring (in time) projected multi-slit patterns. A wide-field image of the eye is acquired as well during the examination session. During the image processing step, the slits are extracted from each of the multi-slit images and each extracted slit is pasted on the wide-field image which serves as the background image. Thus, multiple reconstructed single-slit images are generated from a smaller set of acquired images illuminated by a multi-slit projection and a “background” image (ROI illuminated). A single slit/multiple single slits pasted on top of the background image of the eye emulate a single slit scan (or scan with several slits simultaneously), similar to the eye-physician scan while working with the convention systems.

In the simplest embodiment, the acquired series of multi-slit patterns, i.e., the originally acquired images, may be displayed, and scrolling between a sequence of such images for coverage of all the region of interest may be enough to localize any abnormalities within the region of interest.

In some embodiments, the system will capture a multi slit set, and single slit sets, and upon pointing the slit in the multi-slit image, a single slit (from the other sequence acquired) will be presented to the viewer. In order to detect the correct location of the slit pattern matching methods, eye segmentation and mapping, or multi-to-single slit operation described hereinafter can be used.

In order to separate the slits in the multi-slit image (for use as single slits), the technique of the present disclosure can use either image processing known in the art, such as binarization of the image, foreground and background separation, or semantic segmentation (using DNNs) or a combination of them. Following the foreground background separation and slit segmentation, the slits are cropped from the multi-slit image and pasted on the ‘background image’, and then the slits are ordered/arranged as a sequence of single slits by their location order.

In the posterior part of the eye, and in frontal slits projection and imaging the inventors have found that if the optical projector is operable to sequentially project first and second sets of the illumination spots of a multi-slit shape onto, respective, first and second sets of the locations, wherein the first and second locations are arranged in an interlaced fashion, better image quality of reconstructed single-slit images can be obtained. This interlaced method of arranging the first and second locations is called herein an “even/odd method” and is used for retinal multi-slit imaging or full-frontal imaging (without tilting angles), based on a multi-slit projection.

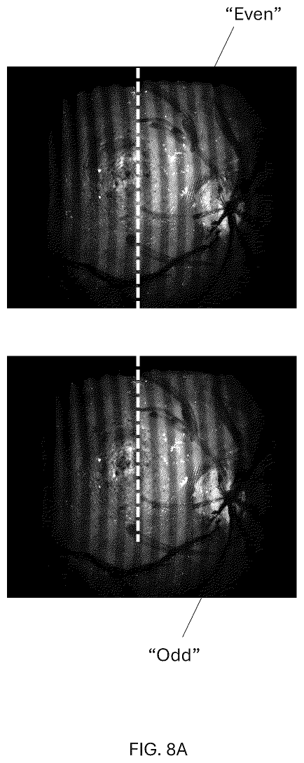

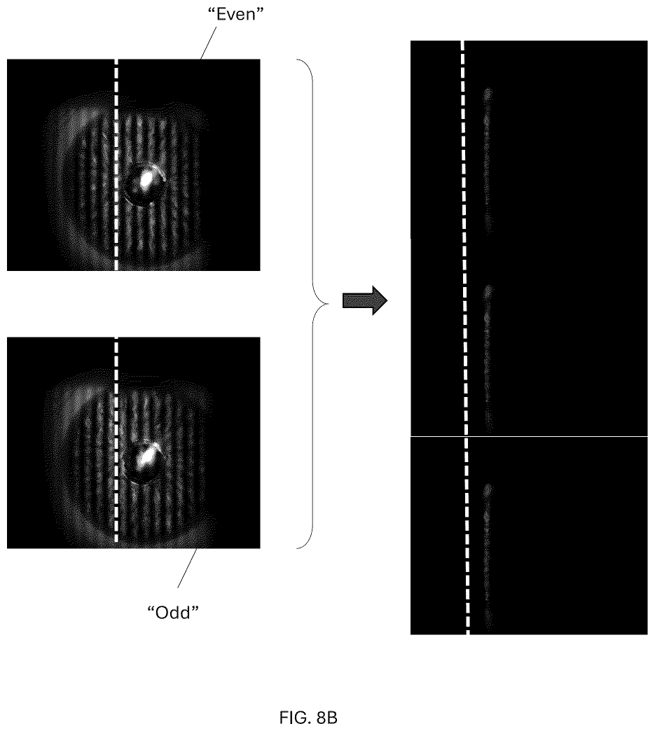

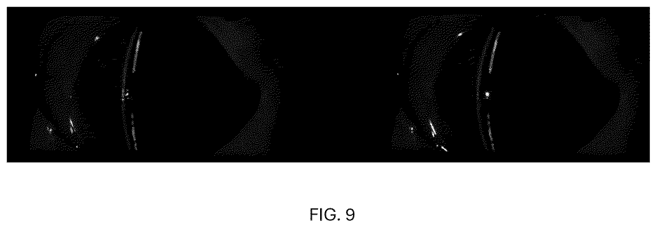

FIGS. 8A and 8B exemplify an acquisition method including capturing two multi-slit pattern projections, “even” and “odd”, where the “odd” projection includes inverse illumination to the “even” projection, i.e., dark (in the “odd” set) where bright was (in the “even” set), and bright (in the “odd” set) where dark was in the “even” set). A dashed white line drawn on the two “even” and “odd” interlaced multi-slit images helps to understand the interlacing principle.

An additional, third, “background” image might be captured as well. The interlaced multi-slit projections may include non-overlap slits where the two projections (“even” and “odd”) can or cannot overlap each other, namely, the sum of the alternating projections may create a continuous coverage if so required or left on purpose with gaps. The slits of the “even” and “odd” patterns may cover all the field of view, such that if the two projections are simultaneously illuminated, it is equivalent to illuminating the entire field-of-view with full illumination. The projection angle and the slit width and space between the slits can be correlated, to create the multi-slit series. Typically, the interlaced (i.e., “even” and “odd”) images are subtracted from each other in order to obtain better contrast.

The methods used for slit separation during image processing may include one or more of the following image processing methods: adaptive image binarization, foreground binarization using for example Otsu method, foreground cropping. Additionally, known in the art semantic segmentation methods, e.g., deep neural network (DNN) methods may be used as well. Labeling is done using standard image processing methods, such as Watershed methods and Enhanced Watershed image processing methods known in the art. A DFS (Depth First Search) algorithm may be applied to collect the slits in their projection order.

During reconstruction, single/multiple separated slits may be pasted on top of a wide-field background image of the eye.

In the embodiments where traditional slit-scan emulation is required, the generated images are reconstructed by pasting each one of the slits from each one of the “even” and “odd” multi-slit images on top of a background image. This method provides a complete coverage of the ROI and when selected during the examination session, allows single-slit scan emulation by displaying the image with the slit on the current scan location. In this method, due to the even-odd nature, only 3 images may be used (even, odd, background) in order to cover all the regions of interest. This may result in a very short acquisition process of less than saccade movement, i.e. when capturing the posterior part of the eye, resulting in very accurate imaging.

This method of reconstruction may also be used when images are acquired by two cameras simultaneously, allowing thus a stereo vision slit scan, as with conventional slit-lamp methods. Such stereo vision slit scan results in a video-like slit-scan of the examined area of the eye, similar to scans obtained via the traditional slit lamp device. The scan can be stopped/repeated/played from end to start, thus changing the direction of the scan, change playing speed as in a captured video.

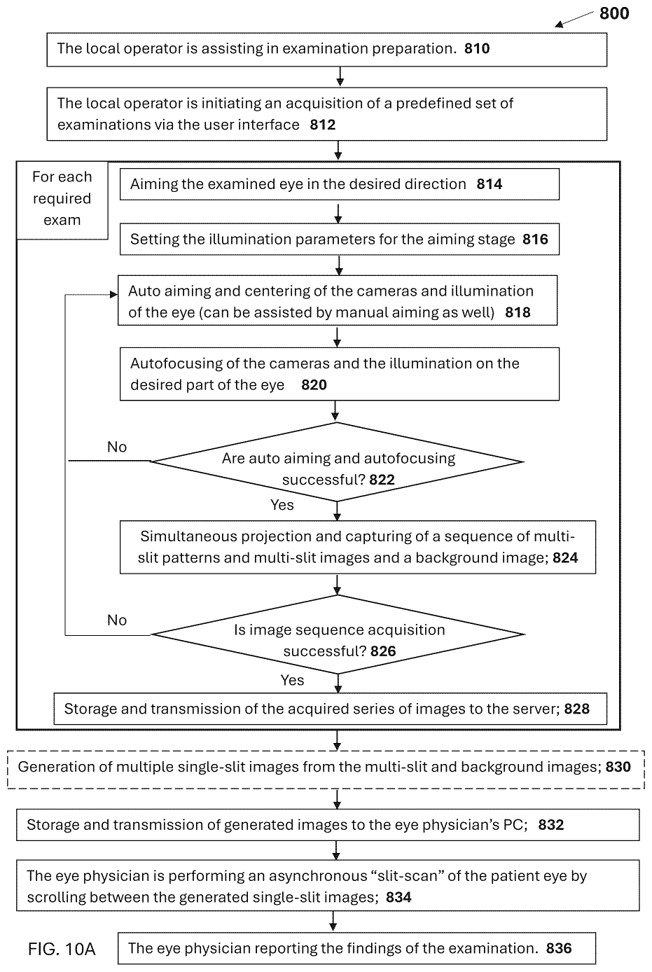

FIG. 9 shows a stereo single-slit view of the examined eye, as displayed to the remote eye-physician. Each slit was taken from its respective angle (as if the physician views it via the eyepieces of the slit lamp). The two images are interpreted by the eye-physician as a stereo single-slit image, similar to examination with the traditional, binocular slit lamp device.

In some embodiments, the physician can view the multi-slit that was captured simultaneously (from right and left camera) and view the multi-slit in stereo view.

In some embodiments, the method of obtaining multiple reconstructed single-slit images from a smaller set of multi-slit images may be applied also for non-frontal slits of any part of the eye, including the anterior segment where the slits can be observed on multiple layers, surfaces, and depths of semi-transparent layers. In these embodiments, a number of slit pattern projections is/are captured, with a constant and/or variant space between the slits. The patterns may be non-overlapping between slits of different patterns or have partial overlap (for example, for getting smoother and continued scanning by the physician during the slit-scan stage, later in the examination procedure). In some embodiments, the slits may cover all the field-of-view or some region of interest (ROI) for the current exam. The aggregation of all the slits from all the projected patterns, acquired during a fast acquisition of a sequence of multi-slit images, while they stayed at the same/similar location, will cover the entire ROI, so that almost every location of the ROI will be covered by a slit. The reconstruction may include pasting single/multiple separated slits on top of a background image, as described above.

In case of traditional slit-scan emulation use-case, the generated images are reconstructed by pasting each of the slits from each of the multi-slit images on top of a background image, providing thus a complete coverage of the ROI. When single-slit scan emulation is requested e.g., by the physician, the reconstructed image may be displayed with the slit on the current scan location.

It should be noted that the projected pattern of illumination spots, e.g., having a shape of a slit, should provide a sufficient space between its consecutive spots/slits on the captured image of the eye. The space between the spots/slits may be defined according to other system parameters of the acquired examination, i.e. slit width, projection angle, specific ophthalmic condition, etc. In anterior segment examination, each slit projection on the cornea shall not overlap with the consecutive slit projection on the iris.

This condition of non-overlap applies also for cornea/iris with projection on the lens, and corneas or irises of consecutive slits (may be in case of illumination from big angle). For example, capturing the eye image using the illumination directed/propagating along an axis forming an angle of about 60 degrees from the right side with respect to the eye optical axis results in a situation where the projection on the right cornea appears to the left of the iris reflections.

In some embodiments, single-slit images can be used as part of the acquisition sequence, for example, to overcome separation of challenging slit images, difficult for the machine to decipher. This can occur in cases such as saturation, blindness, Iris boundary etc. Multi-slits can be used without projecting the specific slit, that is taken in the single-slit image separately. The multi-slit images might be broken down into separate single-slit images and added to single-slit images for the problematic places for one continuous series. In this case, a sequence of multi-slit images and a sequence of single-slit images are acquired (at the same FOV), and the single-slit images are used in order to separate single slits from the multi-slit images. In some embodiments, a combination of multi-slits and single slits set can be used during the same acquisition or in consecutive acquisitions.

In these embodiments, more images may be required than in a standard sequence of multi-slit projection, and the image analysis is similar to the above-described “even/odd” method.

In the above-described examples, the physician is viewing the images in 3D. The present disclosure also provides an alternative approach of using 3D reconstruction, i.e. creating the solid mesh of an eye, using the slits as if, for example structured light, while preserving and presenting the slits as a texture on this solid. Thus, three-dimensional single slit images rendering method for the anterior or posterior segments of the eye can be used. This method enables reconstruction of the three-dimensional structure of the examined part of the eye, acquired by the multi-slit sequence pattern projection, followed by rendering of the required slit and the background. The slits may be generated in sequential order thus allowing an emulation of dynamic scan of the object/eye.

Such 3D reconstruction can be used in one or more of the following: (1) It can assist in slit separation of the multi-slit and in particular the slit projection on the cornea. Since the 3D location of the cornea is on a higher surface (closer to the camera) than the iris or eye lens. (2) the 3D reconstruction provides 3D information of the slits. (3) Since in any case of 3D reconstruction a calibration process is performed, the 3D reconstruction actually provides physical measurements of the findings (due to the transformation of pixel to inch/mm as part of the calibration). (4) 3D reconstruction provides emulation view of the eye without the need of virtual reality (VR) or 3D screen.

The present disclosure provides methods for measuring elements in the eye, e.g., distances (two and three-dimensional) by using the eye structure extracted from image data provided by at least one camera. The three-dimensional structure may be analyzed using methods known in the art. For example, the three-dimensional structure of the eye may be obtained from structured light projection even when using only single camera with the slit projection. Structure analysis may utilize three-dimensional stereo vision when the imaging device includes at least two cameras and/or when three-dimensional structured light is combined with stereo vision for higher accuracy.

Additionally, 3D reconstruction of the acquired image data may be used to obtain measures of [pixel/mm] as part of 3D calibration process, which may be used during analysis of eye structures extracted from the acquired image data.

The techniques of the present disclosure allow a single acquisition of sequence of multi-slit patterns or by using other structured light or any other method The three-dimensional image/eye reconstruction of the image data resulting from any possible slit-scan and with any examination parameters can be conducted, even at a later date. In some embodiments, slit separation is performed by using the three-dimensional shape of the slits as captured.

In some other embodiments, a point cloud of the slits is built using methods known in the art, such as structured light (if using single camera) or by stereo vision (if using more than one camera) or a combination of the two. Slit analysis and rendering is made using the depth map derived from the points cloud using methods known in the art. The depths of any eye part such as sclera, iris, cornea, or retina are determined by the depth map from the points cloud. It is noted that the projection direction relative to the eye determines the slits order as projected.

Generation of multiple reconstructed single-slit images from a smaller set of acquired images illuminated by a multi-slit projection and a “background” image may utilize semantic segmentation using DNN (Deep Neural Network) known in the art techniques, such as SegNet, UNet, etc. These techniques are considered as methods to analyze the image data and produce the final three-dimensional map.

In general, image quality can be measured through convolutional neural network (CNN) classification or other methods known in the art.

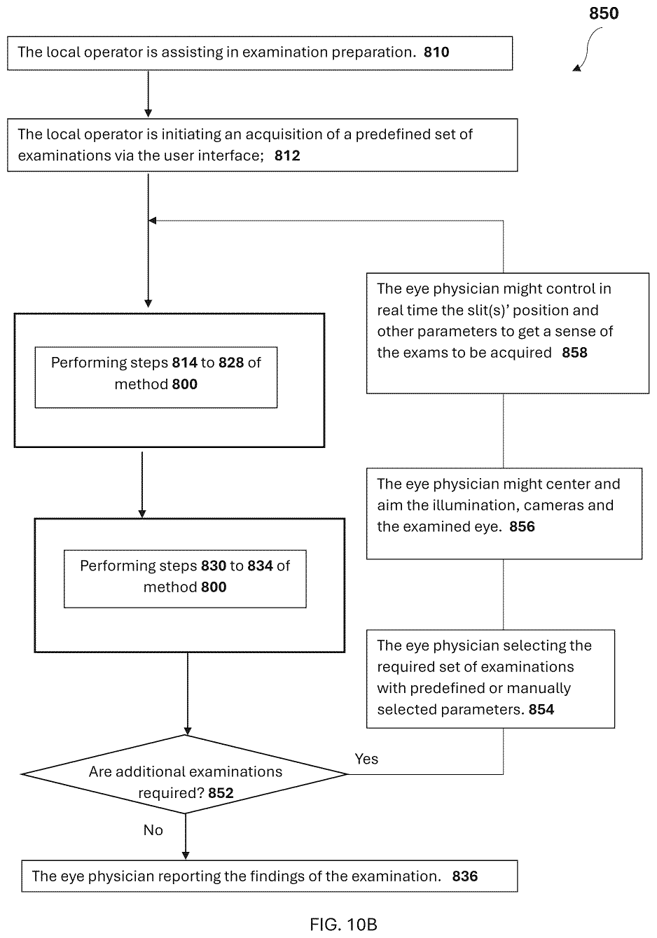

FIG. 10A is a flow diagram exemplifying an asynchronous examination procedure 800 using the eye examination system of the present disclosure, including a fast projection (and respective acquisition of images) of multi-slit patterns. It should be noted that the procedure exemplified in this figure can be also used with single-slit set acquisition, or a combination of those methods as described above.

The procedure starts (step 810) with the local operator assisting the patient in examination preparation, for example, positioning his/her head on the chinrest. Next (step 812), the local technician initiates the examination program.

As described above, one or more presets (predefined examination) are available for selection through the user interface, e.g., the technician's screen 176 communicating with the manager utility 155. Generally, a set of pre-defined exams (related to herein as “presets”) to be performed by default is stored in the memory 153 or retrieved dynamically from the server of the control system 150 and may be initiated by the local operator. Examples of presets selected/instructed by the physician may include and are not limited to cornea, fluorescein, contact lenses fitting, anterior chamber, Iris and posterior chamber, retro-illumination, lens/IOL (intraocular lens), adnexa, eye movements, pupil reaction to light.

The stored presets may be specific for each clinical exam (e.g., directed to detect\analyze specific pathology). Each preset may include and is not limited to pre-defined values of illumination angle, imaging angle, slit shape and intensity, etc. In addition, the semi-autonomous system 110 of the present disclosure may be focused to the optimal point in the eye as required by the specific exam. To this end, the system utilizes a novel focusing mechanism which is based on the understanding of both eyes' anatomy, clinical exam requirements, and system adjustment. For example, if the exam is directed to the cornea (which is a transparent object), the preset may give a command to initially focus on the iris or blood vessels on the cornea (which provides a high contrast) and then move the system's focus from the iris' focal point backward toward the requested focal point on the cornea, which can be easily estimated based on average sizes of a human eye (e.g., about 6 mm backwards).

In some embodiments, the preset may include a specific illumination pattern and color which may assist to detect contact lenses and the marking over them.

Specifically, the preset parameters may include one or more of the following and are not limited to: Desired eye (Left/Right/Both); Initial HW positions (for all/some degrees of freedom)—might also include chinrest height; Fixation Target (eye aiming/ gaze target)—might be variable during the acquisition (for each frame); Background Illumination—might be multispectral parameters, might be variable during the acquisition (for each frame); Region of Interest; Optical/Digital Zoom; Aperture control for camera/s and illumination source/s lenses; Autofocus Target/s (including final offset fix); Lens Selection (in case of several interchangeable lenses); Lens parameters (in case Zoom and/or focus can be internally controlled); Number of acquisitions (sets with the same preset); Thresholds for image quality (minimal required grade/s); Spots shapes including contours for each pattern to be projected/binary map for each pattern; Safety HW limits parameters.

In case of sequence of slits/multi-slits being used, the present parameters may include one or more of the following: slit parameters (such as exposure duration; width; height; offset (of the leftmost slit in the pattern); number of slits in pattern/single—multi-slit selection; space (between consequential slits in case of multi-slit); rotation angle; tilt; acquisition (projector) illumination—might be multispectral parameters (additional illumination), might be variable during the acquisition (for each frame/pattern); illumination duty cycle (%) of the slit segment on each part of the eye (for example: cornea=100%, sclera=45% (would fix the saturation issue on the sclera-see example of FIG. 4C); camera(s) parameters which might be adjustable for each frame and which include one or more of exposure duration; gamma; gain (for each (color) channel and master gain); black level.

It should be noted that the manager utility 155 of the semi-autonomous system 110 of the present disclosure enables the system to function autonomously, e.g., during the asynchronous examination 800, in which the local operator can simply press a button to initiate the series of tests (presets), the results of which will sent for analysis by a physician.

The examination plan guides the technician to aim the examined eye in the desired direction (step 814) and to set the illumination parameters for the aiming stage (step 816). Auto aiming and centering of the cameras and illumination of the eye (can be assisted by manual aiming as well) is performed in step 818. The correct location of capturing/imaging unit and the illumination unit are prescribed by the examination plan. Autofocusing of the cameras and the illumination on the desired part of the eye is performed in step 820. These steps (818 and 820) can be done either manually or automatically or by a combination of both. Positioning and other operations (described hereinafter) may be performed using IR/“soft” or ambient illumination in order to ease the patient's comfort. The automatic operations prior to image capture, e.g., steps 818 and 820, may be applied iteratively, i.e. if auto-aiming and autofocusing are not successful (step 822) steps 818 and 820 may be repeated. The control software may control the DOF of both the illumination and the image capture independently and may be parametrized according to the case.

Thus, the automatic operations of the semi-autonomous system prior to image capture may include one or more of the following: auto-aiming of gaze direction of the examined/second eye, auto-centering of the examined eye, initial auto-focus, auto-illumination, final auto-focus. Once the image sequence acquisition is successful (checked in step 824 by estimation of the local operator, remote OD or using image quality method, e.g., CNN or using image processing methods), in step 826, the acquired (one or more) images are stored (e.g., in memory 153) sent to the server (step 828) for generation/reconstruction of (not limited to) multiple single slit images (step 830).

The steps 814 to 828 are performed for each required exam, as indicated in the pre-defined set of examinations obtained by the local technician in step 812. The acquired/generated/reconstructed single-slit images are stored (stored locally or remotely, e.g., in the cloud server, which as noted above, can be implemented in an actual cloud or even locally or on a remote computer), and sent to the PC of the eye-physician for evaluation (step 832). The eye physician performs an asynchronous “slit-scan” of the patient eye by scrolling between the generated single-slit images (step 834) and reports the findings of the examination session (step 836). The eye physician may choose to display the results as multi-slit image (if the multi-slit image acquisition mode as shown as optional step 830) or single-slit (after algorithmic separation) images. The display to the eye physician may include any one of the following: a two- and/or three-dimensional display, such as virtual reality, deep reality viewer, three-dimensional screens, three-dimensional modeling (three-dimensional mesh with texture) of images, or reconstructed from images, acquired from different angles.

FIG. 10B is a flow diagram exemplifying a synchronous examination procedure 850 using the eye examination system of the present disclosure, including a fast projection (and respective acquisition of images) of multi-slit patterns. The procedure 850 is similar to procedure 800 of FIG. 10A in all respects, except for the possibility given to the eye physician to decide in real time whether to retake the examination with the same or different examination parameters.

For example, the eye physician may decide that additional examinations are required (step 852). In this case, several non-limiting actions may be taken: (i) the eye physician may select the required set of examinations with predefined or manually selected parameters (854); (ii) the eye physician might center and aim the illumination, cameras and the examined eye using the remote control capabilities of the semi-autonomous system of the present disclosure (856); the eye physician might control in real time the slit(s)' position and other parameters to get a sense of the exams to be acquired (858). Non-limiting examples of controlled exam parameters include: slit width, slit height, RGB (wavelength), illumination intensity, illumination angle, photography angle, focus offset, eye steering, background illumination.

The system for eye examination of the present disclosure may further include various human machine interface features (HMI) which are not shown in the figures, e.g., deployment of a touch screen, joystick or keyboard of the virtual reality hardware for control of the various system components, visual instructions (e.g., for examined or second eye aiming), vocal instructions (for initiation of the examination, documentation of the procedure/findings/etc. by the physician as well as automated instruction of the system to the patient). Additionally, the eye examination of the present disclosure may further include means for remote/local definition of investigated area and for remote/local request for zooming and focusing on the required area.

It should be noted that the above flow diagrams show specific but not limiting examples of the technique of the present disclosure.

It should also be noted that in some embodiments the proper AI-based data analysis can be used that may point to suspicious areas or findings, pathologies recognition and alerting, marking suspicious areas, coloring physician findings, auto measurements of pathologies marked by the physician (radius, variation, etc.), upon findings auto centering and aiming and/or capturing with specifications relevant to these findings, etc.

The examination procedure may utilize different illumination intensities relative to the eye region/location being imaged: for example, lower illumination intensity for imaging sclera region and higher illumination intensity for imaging the corneal region, on the same slit or projection. In another example, the illumination is properly adjustable. For example the illumination is adjusted based on the color of the iris or modified according to the characteristics of the examined area (e.g., a nevus, which may be darker than the surrounding area). It is noted that the illumination adjustments can be performed automatically and as part of an autonomous process.

1. A system for eye examination comprising:
a semi-autonomous system configured and operable to perform examination sessions of the eye, the semi-autonomous system comprising:
an illumination device comprising at least one optical projector, each of the at least one optical projector being configured and controllably operable to automatically project a sequence of illumination spots of a predetermined shape on a plurality of locations extending in a spaced-apart relationship across an eye region during the examination session of the eye; and
at least one imaging device, each of the at least one imaging device being configured and operable to acquire images of the eye comprising said plurality of locations being illuminated by the illumination spots during the examination session and generate image data in association with the plurality of locations, said image data being indicative of abnormalities with respect to each said locations within the eye region, and
a control system comprising a manager utility and a controller, wherein said manager utility is configured and operable to be responsive to input data comprising preset data indicative of an examination task to define operational data for the semi-autonomous system being controlled by said controller, said operational data defining operational parameters of the examination session of the eye, said controller being configured to operate each of said at least one imaging device synchronously with a respective at least one optical projector thereby enabling to minimize image deterioration factor in said image data and minimize time of the eye exposure to the illumination during the examination session.

2. The system according to claim 1, wherein the input data bas at least one of the following configurations:
the preset data comprises at least one of the following: pre-stored data indicative of the examination task; and dynamically provided input from a physician at a remote physician-related station comprising data indicative of the examination task;
the preset data comprises a list of records in association with at least one of a specific pathology type, functionality, and examination of a specific part of the eye region, said records comprising a plurality of data records relating to various types of the operational data to be used in the examination session;
the preset data comprises data indicative of two or more examination tasks across different regions of the eye, the control system being configured to operate the semi-autonomous system in a fully automatic mode enabling automatic execution of a sequence of two or more examination tasks, thereby providing data indicative of broad overview of the eye condition;
the preset data comprises a plurality of patients' identification data in association with prescribed operational data;
the input data comprises data being received from a remote station of a physician during physician's review of image data provided by the control system, thereby enabling the physician preview of expected image acquisition during the examination session and enabling the physician to provide various examination parameters;
comprising data being received from a remote station of a physician being indicative of physician preview data of expected image acquisition during the examination session, based on physician's review of initial image data provided by the control system, the physician preview data comprising data indicative of one or more examination parameters.

3. (canceled)

4. (canceled)

5. (canceled)

6. (canceled)

7. (canceled)

8. The system according to claim 1, characterized by at least one of the following:
the control system is configured and operable to generate an activation signal to the semi-autonomous system to start execution of the examination session, while avoiding any movement of any physical element of the semi-autonomous system after generation of the activation signal;
the control system further comprises an image processor utility configured and operable to analyze the image data and generate data indicative of said number of abnormalities in the respective number of locations;
the control system is configured and operable to communicate data indicative of the image data to a remote control station for further processing to identify and analyze abnormalities in the eve;
the control system is configured and operable to control automatic variation of one or more of the following operational parameters independently; dimensions of the illumination spot, illumination wavelength illumination intensity, illumination angle, imaging angle, focus offset of the illumination and/or imaging device, eye steering, background illumination;
the control system is responsive to input data from updated preset data from a suggested AI-based processor and configured to utilize the updated present data to optimize the operational data for the semi-autonomous system;
the control system is configured as an electronic unit to be installed in the semi-autonomous examination system.

9. The system according to claim 1, wherein the semi-autonomous system has at least one of the following configurations:
is configured and operable to perform electronic scan of the eye region with the illumination spots, forming different or varying patterns, being projected on the eye region during the examination session, while keeping physical stationarity of the semi-autonomous system during the examination session;
comprises & focusing mechanism configured and operable to initially focus at least one of the optical projector and the imaging device on a relatively high contrast part of the eye and then move the focus forward or backward toward area part of the eye which is to be examined, based on a known distance between the relatively high contrast part and the part to be examined;
comprises an autofocus mechanism being automatically operated by said controller according to said operational parameters of the examination session of the eye.

10. The system according to claim 1, wherein the illumination device comprises a number N (N≥1) of the optical projectors, and a number M (M≥1) of the imaging devices, the numbers N and M being the same or different.

11. The system according to claim 10, wherein the illumination device has one of the following configurations:
comprises at least one pair of the optical projectors, each of the at least one pair of the optical projectors being associated with one or two imaging devices for performing at least one of (i) concurrent examination of both eyes of a patient, or (ii) performing stereo imaging of the eye;
comprises two or more of the optical projectors configured to operate with different operational parameters to concurrently or sequentially perform examination of different parts of the eye region.

12. (canceled)

13. The system according to claim 1, having at least one of the following configurations:
said at least one optical projector and said at least one imaging device are configured with an extended depth of field, such that the illumination spots being automatically projected on the plurality of locations are focused on the respective plurality of locations, and the image data is indicative of respective focused images of said locations;
said at least one optical projector and said at least one imaging device are configured with an extended depth of field independently controlled by said controller;
said at least one optical projector is configured and operable to automatically vary a spherical angle of projection of the illumination spot with respect to the eye, the image data comprising image data pieces of the respective locations in association with the data indicative of the spherical angle of projection.

14. The system according to claim 1, wherein said at least one optical projector and said at least one imaging device are configured with an extended depth of field independently controlled by said controller, said extended depth of field being selected to include multiple anterior segment parts of the eye and adnexa being in focus at once.

15. (canceled)

16. (canceled)

17. The system according to claim 1, wherein at least one of the optical projector and the imaging device comprises an autofocus mechanism being automatically operated by said controller according to said operational parameters of the examination session of the eye.

18. (canceled)

19. The system according to claim 1, wherein said predetermined shape of the illumination spot is a slit-like shape.

20. The system according to claim 19, having one of the following configurations:
said at least one optical projector is configured and controllably operable to automatically project said sequence of slit-like shaped illumination spots in a single-slit consecutive fashion, said image data comprising corresponding consecutive image data pieces, each image data piece comprising a single-slit image;
said at least one optical projector is configured and controllably operable to automatically and simultaneously project at least a first array of spaced-apart slit-like shaped illumination spots in at least first multi-slit fashion, said image data comprising at least one first image, each being image of the at least first array of spaced-apart locations.

21. The system according to claim 19, wherein said at least one optical projector is configured and controllably operable to automatically and simultaneously project at least a first array of spaced-apart slit-like shaped illumination spots in at least first multi-slit fashion; said image data comprising at least one first image, each being image of the at least first array of spaced-apart locations, said optical projector being operable to sequentially project said first array of the slit-like shaped illumination spots in the first multi-slit fashion and a second array of the slit-like shaped illumination spots in a second multi-slit fashion, on respective first and second arrays of the locations, wherein the first and second arrays of the locations are arranged in an interlaced fashion; said image data comprising the first and second images of respectively the first and second arrays of locations.

22. (canceled)

23. The system according to claim 21, wherein said first and second locations are partially overlapping.

24. (canceled)

25. The system according to claim 1, further comprising at least one of the following: an imaging unit configured and operable to obtain a wide-field image of the eye, to thereby enable to align foreground image formed by said image data on a background image formed by the wide-field image of the eye; and a registration assembly for registering a position of user's face during the examination session.

26. The system according to claim 1, further comprising a registration assembly for registering a position of user's face during the examination session, the system being characterized by at least one of the following:
said control system further comprises an activation utility configured and operable to activate the semi-autonomous system to perform the examination session in response to a control signal indicative of a safety condition of the user's face being at the registered position;
the system further comprises a sensing system configured and operable to monitor user's face position and generate corresponding sensing data to be analyzed in order to determine the user's face position with respect to the registered position for selective generation of the control signal.

27. (canceled)

28. The system according to claim 26, further comprising a sensing system configured and operable to monitor user's face position and generate corresponding sensing data to be analyzed in order to determine the user's face position with respect to the registered position for selective generation of the control signal, the system being characterized by at least one of the following:
said sensing system is configured and operable to communicate the sensing data to a safety controller at a remote control station, where the sensing data is analyzed, and the control signal is selectively generated and communicated to the controller;
the system further includes a safety controller being responsive to the sensing data and configured and operable to analyze the sensing data and generate the control signal to said controller upon identifying the registered position of the user's face.

29. (canceled)

30. (canceled)

31. The system according to claim 1, wherein said illumination device comprises a flash illuminator.

32. The system according to claim 1, wherein said semi-autonomous system comprises at least one of the following:
an eye-aiming device automatically operated by said controller according to said operational parameters of the examination session of the eye;
an eye tracking mechanism operated automatically by said controller during said examination session;
one or more movement mechanisms for implementing controllable movement of one or more elements of the system; and comprises a safety controller configured and operable to control said one or more movement mechanisms.

33. (canceled)

34. (canceled)

35. The system according to claim 1, wherein-characterized by at least one of the following: the at least one imaging device is configured and operable to capture 3D images said at least one imaging device comprises color or black and white sensors; and said at least one imaging device comprises monocular and/or multi-spectral sensor.

36. (canceled)

37. (canceled)

38. (canceled)

39. (canceled)

40. The system according to claim 19, wherein said at least one optical projector is configured and controllably operable to automatically project said sequence of slit-like shaped illumination spots in a single-slit consecutive fashion, said image data comprising corresponding consecutive image data pieces, each image data piece comprising a single-slit image, the control system further comprising an image processor utility configured and operable to extract said single slit image out of each of said consecutive image data pieces of the respective locations, crop said extracted single slit image and paste said cropped extracted single slit image on top of a background image being a wide-field image of the eye.

41. The system according to claim 19, wherein said at least one optical projector is configured and controllably operable to automatically and simultaneously project at least a first array of spaced-apart slit-like shaped illumination spots in at least first multi-slit fashion, said image data comprising at least one first image, each being image of the at least first array of spaced-apart locations, the system being characterized by at least one of the following:
the control system further comprising an image processor utility configured and operable to segment said at least first multi-slit image into a set of corresponding single-slit images;
the semi-autonomous system is configured and operable to perform stereo imaging of the eye and provide stereo image data of the eye region, and wherein said image processor is configured and operable to utilize said stereo image data to generate a 3D reconstructed image out of two multi-slit images; identify at least one 3D single-slit image in said 3D reconstructed image; project said at least one 3D single-slit image onto a 2D image thereby obtaining a 2D projected image comprising at least one single-slit image, extract said at least one single slit image out of said 2D projected image; crop said extracted single slit image; and paste said cropped extracted single slit image on top of a background image being a wide-field image of the eye.

42. The system according to claim 41, wherein the image processor utility has at least one of the following configurations: is further configured and operable to paste each of the single-slit images, extracted from said at least first multi-slit image, on top of a background image being a wide-field image of the eye; and is configured and operable to generate data indicative of dynamic movement of the single-slit image over the eye, thereby enabling human passing of the data being presented for a specific slit view.

43. (canceled)

44. The system according to claim 1, wherein the control system further comprises an image processor utility configured and operable to analyze the image data and generate data indicative of said number of abnormalities in the respective number of locations, said image processor utility having at least one of the following configurations:
the image processor utility is configured and operable to perform at least one of the following: utilize the image data to generate 3D reconstruction of the acquired data in said acquired images;
the image processor utility is configured and operable to analyze said image data by applying algorithm techniques including one or more of artificial intelligence, image enhancement, image recognition, image sharpening and image restoration encoding;
the image processing utility is integral with at least one the optical projector and the imaging devices.

45. The system according to claim 19, wherein said at least one optical projector is configured and controllably operable to automatically and simultaneously project at least a first array of spaced-apart slit-like shaped illumination spots in at least first multi-slit fashion, said image data comprising at least one first image, each being image of the at least first array of spaced-apart locations, the semi-autonomous system being configured and operable to perform stereo imaging of the eye and provide stereo image data of the eye region, and wherein said image processor is configured and operable to utilize said stereo image data to generate a 3D reconstructed image out of two multi-slit images; identify at least one 3D single-slit image in said 3D reconstructed image; project said at least one 3D single-slit image onto a 2D image thereby obtaining a 2D projected image comprising at least one single-slit image; extract said at least one single slit image out of said 2D projected image; crop said extracted single slit image; and paste said cropped extracted single slit image on top of a background image being a wide-field image of the eye, the illumination device comprising at least one pair of the optical projectors, each pair of the optical projectors being associated with one or two imaging devices for performing said stereo imaging of the eye.

46. (canceled)

47. (canceled)

48. (canceled)

49. The system according to claim 1, wherein said manager utility is configured for data communication with an image processor at a remote control station.

50. The system according to claim 49, characterized by at least one of the following: said data communication comprises data transfer using encrypted secure communication; and said manager utility is configured and operable to generate compressed representation of said image data and transfer said compressed representation to the image processor at the remote control station.

51. (canceled)

52. (canceled)

53. (canceled)

54. A control system for managing and controlling eye examination by a semi-autonomous examination system comprising at least one optical projector operable to automatically project a sequence of illumination spots of a predetermined shape on a plurality of locations extending in a spaced-apart relationship across an eye region during the examination session of the eye, and at least one imaging device operable to acquire images of said plurality of locations during the examination session and generate image data in association with the plurality of locations; the control system being configured as a computerized system having data input and output utilities, memory, and an image processor utility, wherein the control system comprises:
a manager utility configured and operable to be responsive to input data comprising preset data indicative of an examination task to define operational data for the semi-autonomous system, said operational data defining operational parameters of the examination session of the eye, and
a controller being configured to utilize said operational data and operate said at least one imaging device synchronously with said optical projector enabling to minimize image deterioration factor in said image data and minimize time of the eye exposure to the illumination during the examination session.

55. The control system according to claim 54, characterized by at least one of the following:
the control system is configured as an electronic unit to be installed in the semi-autonomous examination system;
the control system is configured and operable to communicate with a remote control station, said manager utility being responsive to instructions from the remote control station to update at least one of the preset data and the operational data.

56. (canceled)

57. A control system comprising a computerized system connectable, via communication network, to subscriber eye examination systems of a type performing semi-autonomous eye examination procedures, the control system being configured and operable to receive input image data from each of subscriber eye examination system and apply model-based processing to the received input image data, and generate output data comprising at least one of the following: (i) data indicative of suspected parts and observations in the eye region being examined to enable detection of abnormalities and/or pathologies; (ii) data indicative of abnormalities and/or pathologies in one or more parts of an eye region being examined by the respective subscriber eye examination system; (iii) data indicative of a quality level of the received image data provided by the respective subscriber eye examination system; and (iv) data indicative of recommended preset data defining operational data for examination sessions to be performed by the respective subscriber eye examination system.
